(19) United States
(12) Patent Application Publication
(10) Pub. No.: US 2019/0337971 A1
(43) Pub. Date: Nov. 7, 2019

(54) PANTETHEINE DERIVATIVES FOR THE TREATMENT OF NEUROLOGIC DISORDERS

(71) Applicant: Retrophin, Inc., San Diego, CA (US)

(72) Inventors: Maria Beconi, Bedford, MA (US); Daniel Elbaum, Newton, MA (US); Savina Malancona, Rome (IT)

(21) Appl. No.: 16/320,443

(22) PCT Filed: Jul. 24, 2017
(86) PCT No.: PCT/US2017/043563

§ 371 Date: Jan. 24, 2019

Related U.S. Application Data
(60) Provisional application No. 62/366,428, filed on Jul. 25, 2016.

(51) Int. Cl. C07F 9/6571; C07F 9/6574; A61P 25/00

(52) U.S. Cl. CPC C07F 9/657154; A61P 25/00; C07F 9/65742

(57) ABSTRACT

Compounds having the following formula (I): Formula I and pharmaceutically acceptable salts thereof, wherein A, B, D, E and R1 are as defined herein, are provided. Methods comprising the use of such compounds for the treatment of neurological disorders, such as pantothenate kinase-associated neurodegeneration, and pharmaceutical compositions containing such compounds, and their use in the treatment of neurological disorders, also are provided.

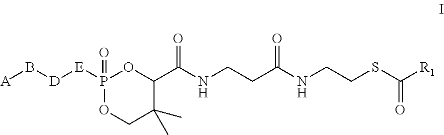

### BACKGROUND

### Technical Field

The present disclosure relates to pantetheine derivatives, pharmaceutical compositions containing such compounds, and their use in the treatment of neurologic disorders (such as pantothenate kinase-associated neurodegeneration).

### Description of the Related Art

Pantothenate kinase-associated neurodegeneration (PKAN) is a form of neurodegeneration with brain iron accumulation (NBIA) that causes extrapyramidal dysfunction (e.g., dystonia, rigidity, choreoathetosis) (A. M. Gregory and S. J. Hayflick, “Neurodegeneration With Brain Iron Accumulation”, Orphanet Encyclopedia, September 2004). PKAN is a genetic disorder resulting from a deficiency of the enzyme pantothenate kinase, which is responsible for the conversion of pantothenic acid (vitamin B5) to 4 ‘-phosphopantothenic acid. 4’-Phosphopantothenic acid is subsequently converted into Coenzyme A (CoA) (as shown below) (R. Leonardi, Y.-M. Zhang, C. O. Rock, and S. Jackowski, “Coenzyme A: Back In Action”, Progress in Lipid Research, 2005, 44, 125-153).

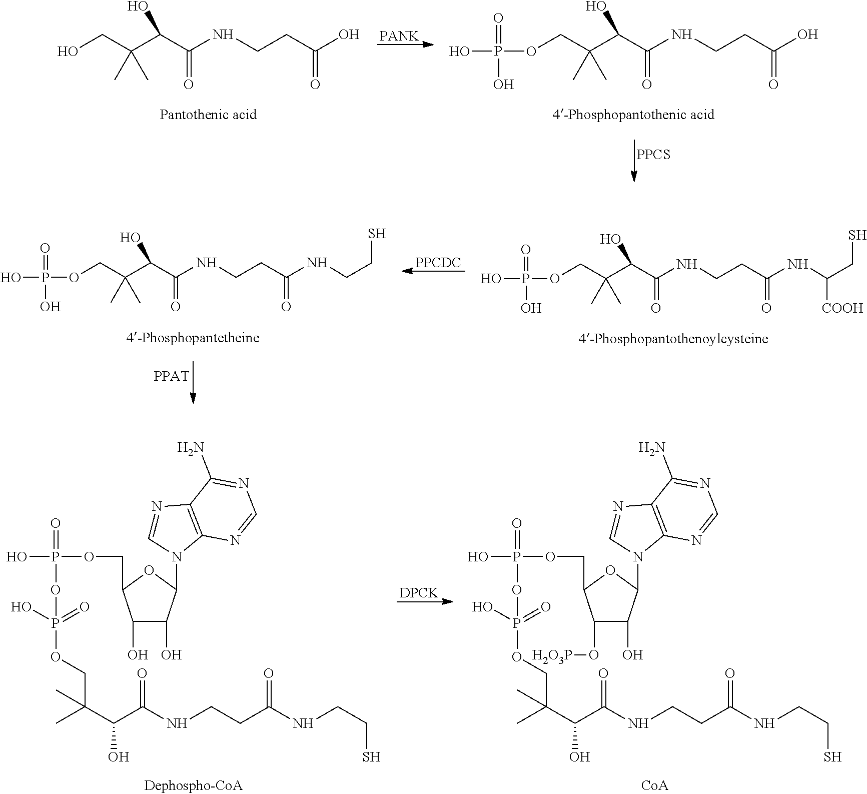

In particular, pantothenic acid is converted to 4′-phosphopantothenic acid via the enzyme pantothenate kinase (PANK), which is converted to 4′-phosphopantothenoylcysteine via the enzyme 4′-phosphopantothenoylcysteine synthase (PPCS), and subsequently decarboxylated to 4′-phosphopantetheine via 4′-phosphopantothenoylcysteine decarboxylase (PPCDC). 4′-phosphopantetheine is then appended to adenosine by the action of phosphopantetheine adenyltransferase (PPAT) to afford dephospho-CoA, which is finally converted to coenzyme A (CoA) via dephospho-CoA kinase (DPCK).

Classic PKAN usually presents in a child's first ten to fifteen years, though there is also an atypical form that can occur up to age 40. PKAN is a progressively degenerative disease that leads to loss of musculoskeletal function with a devastating effect on quality of life.

One approach to treating PKAN could be to administer 4′-phosphopantothenic acid. This approach has been mentioned in the literature, but it has been recognized that the highly charged molecule would not be able to permeate the lipophilic cell membrane (C. J. Balibar, M. F. Hollis-Symynkywicz, and J. Tao, “Pantethine Rescues Phosphopantothenoylcysteine Synthetase And Phosphopantothenoylcysteine Decarboxylase Deficiency In Escherichia Coli But Not In Pseudomonas Aeruginosa”, J. Bacteriol., 2011, 193, 3304-3312).

Thus, there remains a need for compounds useful in treating various diseases, such as PKAN.

### BRIEF SUMMARY

In certain aspects, the present invention is directed to compounds having the following structure (I):

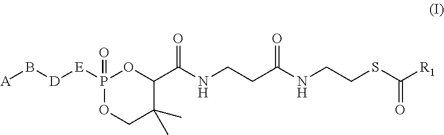

and pharmaceutically acceptable salts thereof, wherein A, B, D, E and R₁ are as defined herein.

In another aspect, the present invention also is directed to pharmaceutical compositions comprising a compound of Formula I, or a pharmaceutically acceptable salt thereof, and a pharmaceutically acceptable carrier, diluent or excipient.

In yet another aspect, a method of treating a subject having a disorder associated with pantothenate kinase enzyme deficiency is provided, comprising administering to a subject in need thereof an effective amount of a compound of Formula I, or a pharmaceutically acceptable salt thereof.

The present invention also provides a method of treating a subject having a disorder associated with Coenzyme A deficiency, the method comprising administering to the subject an effective amount of a compound of Formula I, or a pharmaceutically acceptable salt thereof.

These and other aspects of the present invention will become apparent upon reference to the following detailed description. All references disclosed herein are hereby incorporated by reference in their entirety as if each was incorporated individually.

### DETAILED DESCRIPTION

The instant invention provides pantotheine derivatives, including cyclic pantotheine derivatives. In some embodiments, compounds, pharmaceutical compositions, and methods of use are provided.

In the following description, certain specific details are set forth in order to provide a thorough understanding of various embodiments of the invention. However, one skilled in the art will understand that the invention may be practiced without these details.

Unless defined otherwise, all technical and scientific terms used herein have the same meaning as is commonly understood by one of skill in the art to which this invention belongs. As used herein, certain items may have the following defined meanings.

Unless the context requires otherwise, throughout the present specification and claims, the word “comprise” and variations thereof, such as “comprises” and “comprising,” are to be construed in an open, inclusive sense, that is, as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the specification and claims, “including” and variants thereof, such as “include” and “includes”, are to be construed in an open, inclusive sense; i.e., it is equivalent to “including, but not limited to”.

As used in the specification and claims, the singular for “a”, “an”, and “the” include plural references unless the context clearly dictates otherwise. For example, the term “a cell” includes a plurality of cells, including mixtures thereof. Similarly, use of “a compound” for treatment of preparation of medicaments as described herein contemplates using one or more compounds of the invention for such treatment or preparation unless the context clearly dictates otherwise.

As used herein, “about” and “approximately” generally refer to an acceptable degree of error for the quantity measured, given the nature or precision of the measurements. Typical, exemplary degrees of error may be within 20%, 10%, or 5% of a given value or range of values. Alternatively, and particularly in biological systems, the terms “about” and “approximately” may mean values that are within an order of magnitude, potentially within 5-fold or 2-fold of a given value. When not explicitly stated, the terms “about” and “approximately” mean equal to a value, or within 20% of that value.

As used herein, numerical quantities are precise to the degree reflected in the number of significant figures reported. For example, a value of 0.1 is understood to mean from 0.05 to 0.14. As another example, the interval of values 0.1 to 0.2 includes the range from 0.05 to 0.24.

The term “alkyl” refers to a straight or branched hydrocarbon chain radical consisting solely of carbon and hydrogen atoms, containing no unsaturation.

Unless otherwise specified, the term “alkyl” refers to a group having from one to eight carbon atoms (for example, one to six carbon atoms (i.e., C₁-C₆), or one to four carbon atoms (i.e., C₁-C₄)), and which is attached to the rest of the molecule by a single bond. Examples of alkyl groups include, but are not limited to, methyl, ethyl, n-propyl, i-propyl, n-butyl, t-butyl, s-butyl, n-pentyl, neopentyl and s-pentyl.

The term “alkenyl” refers to an aliphatic hydrocarbon group containing at least one carbon-carbon double bond and which may be a straight or branched chain. Unless otherwise specified, the term “alkenyl” refers to a group having 2 to about 10 carbon atoms, e.g., ethenyl, 1-propenyl, 2-propenyl (allyl), iso-propenyl, 2-methyl-1-propenyl, 1-butenyl, and 2-butenyl.

The term “alkynyl” refers to a straight or branched chain hydrocarbyl radical having at least one carbon-carbon triple bond. Unless otherwise specified, the term “alkynyl” refers to a group having in the range of 2 up to about 12 carbon atoms (for instance, 2 to 10 carbon atoms), e.g., ethynyl, propynyl, and butynyl.

The term “cycloalkyl” denotes a non-aromatic mono or multicyclic ring system of about 3 to 12 carbon atoms such as cyclopropyl, cyclobutyl, cyclopentyl, and cyclohexyl.

The term “cycloalkylalkyl” refers to a cyclic ring-containing group containing in the range of about 3 up to 8 carbon atoms directly attached to an alkylene group which is then attached to the main structure at any carbon in the alkyl group that results in the creation of a stable structure such as cyclopropylmethyl, cyclobutylethyl, and cyclopentylethyl.

The term “cycloalkenyl” refers to a non-aromatic mono or multicyclic ring system of about 3 to 12 carbon atoms and comprising at least one carbon-carbon double bond within the ring system. Examples of cycloalkenyls include, but are not limited to, cyclopropenyl, cyclobutenyl, cyclopentenyl, cyclohexenyl, and the like.

The term “cycloalkenylalkyl” refers to a radical of the form —RₐR_{b}, wherein Rₐ is an alkylene group as defined herein and R_{b} is a cycloalkenyl group as defined herein. Examples of cycloalkenylalkyls include, but are not limited to, cyclopropenylmethyl, cyclobutenylmethyl, cyclopentenylmethyl, or cyclohexenylmethyl, and the like.

The term “aryl” refers to a mono- or multi-cyclic aromatic radical having in the range of 6 up to 20 carbon atoms such as phenyl, naphthyl, tetrahydronapthyl, and indanyl.

The term “arylalkyl” refers to an aryl group as defined above directly bonded to an alkylene group as defined herein, e.g., —CH₂C₆H₅, and —C₂H₄C₆H₅.

The term “heteroatoms” as used herein refers to non-carbon and non-hydrogen atoms, capable of forming covalent bonds with carbon, and is not otherwise limited. Typical heteroatoms are N, O, P, and S. When sulfur (S) is referred to, it is understood that the sulfur can be in any of the oxidation states in which it is found, thus including, for example, sulfoxides (R—S(O)—R′) and sulfones (R—S(O)₂—R′), unless the oxidation state is specified; thus, the term “sulfone” encompasses only the sulfone form of sulfur; the term “sulfide” encompasses only the sulfide (R—S—R′) form of sulfur. When phrases such as “heteroatoms selected from the group consisting of O, NH, NR′ and S,” or “[variable] is O, S . . . ” are used, they are understood to encompass all of the oxidation states of sulfur. Similarly, when phosphorus (P) is referred to, it is understood that the phosphorus can be in any of the oxidation states in which it is found, thus including, for example, organophosphorus compounds including phosphines (R₃P), phosphonates (RP(═O)(OR′)₂), phosphinates (R₂P(═O)(OR″)), phosphites or phosphite esters (R(OR)₃), phosphonites (P(OR)₂R′), phosphinites (P(OR)R′₂), and phosphates or phosphate esters (ROP(O)(OR′)₂), unless the oxidation state is specified. When phrases such as “heteroatoms selected from the group consisting of O, NH, NR′ and P,” or “[variable] is O, P . . . ” are used, they are understood to encompass all of the oxidation states of phosphorus.

The term “heterocyclyl” refers to a non-aromatic 3- to 15-member ring radical, which consists of carbon atoms and at least one heteroatom of nitrogen, phosphorus, oxygen, or sulfur. The heterocyclic ring radical may be a mono-, bi-, tri-, or tetracyclic ring system, which may include fused, bridged or Spiro ring systems, and the nitrogen, phosphorus, carbon, oxygen, or sulfur atoms in the heterocyclic ring radical may be optionally oxidized to various oxidation states. In addition, the nitrogen atom may be optionally quaternized.

The term “heterocyclylalkyl” refers to a radical of the formula —RₐR_{c} where Rₐ is an alkylene group as defined above and R_{c} is a heterocyclyl group as defined above, e.g., —CH₂-heterocyclyl, and —C₂H₄-heterocyclyl.

The term “heteroaryl” refers to an optionally substituted 5- to 14-member aromatic ring having one or more heteroatoms of N, O, or S as ring atoms. The heteroaryl may be a mono-, bi- or tricyclic ring system. Examples of such heteroaryl ring radicals include, but are not limited to, oxazolyl, thiazolyl imidazolyl, pyrrolyl, furanyl, pyridinyl, pyrimidinyl, pyrazinyl, benzofuranyl, indolyl, benzothiazolyl, benzoxazolyl, carbazolyl, quinolyl, and isoquinolyl.

The term “heteroarylalkyl” refers to a radical of the formula —RₐR_{d} where Rₐ is an alkylene group as defined herein and R_{d} is a heteroaryl group as defined above, e.g., —CH₂-hetero aryl, and —C₂H₄-heteroaryl.

When two R groups are said to be joined together to form a ring, it is meant that together with the carbon atom or a non-carbon atom (e.g., nitrogen atom), to which they are bonded, they may furthermore form a ring system. In general, they are bonded to one another to form a 3- to 7-membered ring, or a 5- to 7-membered ring. Non-limiting specific examples are cyclopentyl, cyclohexyl, cycloheptyl, piperidinyl, piperazinyl, pyrolidinyl, pyrrolyl, and pyridinyl.

The term “pantothenic acid” as used herein refers to both the protonated form and the deprotonated form (i.e., pantothenate) of pantothenic acid. Likewise, the term “4′-phosphopantothenic acid” as used herein refers to both the protonated form and the deprotonated form (i.e., 4′-phosphopantothenate) of 4′-phosphopantothenic acid.

By a “ring system” as the term is used herein is meant a moiety comprising one, two, three, or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

The term “spiro-substituted cycloalkyl” refers to a cycloalkyl ring in which two ring atoms are bound to the same atom of the substituted group. Examples of spiro-substituted cycloalkyl groups include the following: 1,1-dimethylcyclopropanyl, 1-methylcyclopentanyl-1-carboxylic acid, and 1-aminocyclopropanyl-1-carboxamide.

The term “heterocyclic ring” refers to a ring system as defined above consisting of carbon atoms and at least one heteroatom of nitrogen, phosphorus, oxygen, or sulfur. The heterocyclic ring radical may be a mono-, bi-, tri-, or tetracyclic ring system, which may include fused, bridged or Spiro ring systems, and the nitrogen, phosphorus, carbon, oxygen, or sulfur atoms in the heterocyclic ring radical may be optionally oxidized to various oxidation states. In addition, the nitrogen atom may be optionally quaternized.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain.

“Cycloalkylene” refers to a divalent cycloalkyl radical.

“Alkylcarbonyl” refers to a radical of the formula —C(═O)Rₑ, where Rₑ is an alkyl group as defined herein.

The term “alkoxy” refers to a radical of the formula —ORₑ where Rₑ is an alkyl group as defined above containing one to twelve carbon atoms. Examples of linear alkoxy groups include but are not limited to methoxy, ethoxy, n-propoxy, n-butoxy, n-pentyloxy, n-hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The term “carbonyl,” refers to a —C(═O)— group.

As used herein, the term “halogen” refers to a fluorine, chlorine, bromine, or iodine atom. As used herein, the term “halo” refers to a fluoro, chloro, bromo, or iodo radical.

“Hydroxy” or “hydroxyl” refers to the —OH radical.

The term “oxo” refers to the ═O substituent.

The term “amino” refers to the —NH₂ radical.

“Hydrazone” refers to the ═N—NH₂ substituent.

“Imino” refers to the ═NH substituent.

“Nitro” refers to the —NO₂ radical.

“Cyano” refers to the —CN radical.

“Thioxo” refers to the ═S substituent.

“Aminoalkyl” refers to a radical of the formula —Rₐ—NR_{f}R_{f} where Rₐ is an alkylene group as defined herein, and each R_{f} is independently a hydrogen, an alkyl group, an aryl group, or a heteroaryl group.

“Alkylamino” and “dialkylamino” refer to radicals of the formula —NHRₑ and —NRₑRₑ, respectively, where each Rₑ is, independently, an alkyl group as defined above containing one to twelve carbon atoms. Examples include, but are not limited to, methylamino, ethylamino, dimethylamino, diethylamino, and the like.

“Alkylaminoalkyl” refers to an alkyl group having one alkylamino substituent. The alkylamino substituent can be on a tertiary, secondary or primary carbon. “Dialkylaminoalkyl” refers to an alkyl group having a dialkylamino substituent.

“Aminocarbonyl” refers to a radical of the formula —C(═O)NH₂.

“Alkylaminocarbonyl” refers to a radical of the formula —C(═O)NRₑRₑ or —C(═O)NHRₑ, where each Rₑ is independently an alkyl group as defined herein. Unless stated otherwise specifically in the specification, an alkylaminocarbonyl group may be optionally substituted as described below.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

Unless stated otherwise specifically in the specification, all of the above groups may be unsubstituted or substituted.

The term “substituted”, unless otherwise specified, refers to substitution with any one or any combination of the following substituents: hydrogen, hydroxy, halogen, carboxyl, cyano, nitro, oxo (═O), thio(═S), alkyl, alkoxy, alkenyl, alkynyl, arylalkyl, cycloalkyl, cycloalkenyl, aryl, heterocyclyl, heteroaryl, —COOR^{x}, —C(O)R^{x}, —C(S)R^{x}, —C(O)NR^{x}R^{y}, —C(O)ONR^{x}R^{y}, —NR^{y}R^{z}, —NR^{x}CO NR^{y}R^{z}, —N(R^{x})SOR^{y}, —N(R^{x})SO₂R^{y}, —(═N—N(R^{x})R^{y}), —NR^{x} C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —SO₂ NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —S O₂R^{x}, and —ONO₂, wherein R^{x}, R^{y}, and R^{z} in each of the above groups can be independently hydrogen atom, alkyl, alkoxy, alkenyl, alkynyl, arylalkyl, cycloalkyl, cycloalkenyl, amino, aryl, heteroaryl, heterocyclyl, or any two of R^{x}, R^{y}, and R^{z} may be joined to form a saturated or unsaturated 3- to 10-member ring, which may optionally include heteroatoms which may be the same or different and are O, N, P, or S.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

The term “subject” refers to a mammal, such as a domestic pet (for example, a dog or cat), or human. Preferably, the subject is a human.

The phrase “effective amount” refers to the amount which, when administered to a subject or patient for treating a disease, is sufficient to effect such treatment for the disease.

The term “dosage unit form” is the form of a pharmaceutical product, including, but not limited to, the form in which the pharmaceutical product is marketed for use. Examples include, but are not limited to, pills, tablets, capsules, and liquid solutions and suspensions.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, “deficiency” of an enzyme refers to the absence of or reduced levels or activity of the enzyme, or the presence of a defective enzyme having decreased activity or function.

As used herein, “deficiency” of a metabolic product refers to the absence of or reduced levels of a metabolic product.

As used herein, “overexpression” of an enzyme refers to an excess in production or activity of the enzyme.

As used herein, “downstream product” of an enzyme refers to a substance the production of which is dependent upon the activity of the referenced enzyme. Similarly, “downstream product” of a compound refers to a substance the production of which is dependent upon the presence of the referenced compound. For example, acetyl coenzyme A (“Acetyl-CoA”) is a downstream product of Coenzyme A.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts that retain the biological effectiveness and properties of the free acids, and are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol (2-dimethylaminoethanol), 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, caffeine, and meglumine.

The invention disclosed herein is also meant to encompass all pharmaceutically acceptable compounds of the structures disclosed herein being isotopically-labeled by having one or more atoms replaced by an atom of the same element having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of structures disclosed herein, for example, those incorporating a radioactive isotope, are useful in drug and/or substrate tissue distribution studies. These radiolabeled compounds could be useful to help determine or measure the effectiveness of the compounds, by characterizing, for example, the site or mode of action, or binding affinity to a pharmacologically important site of action. The radioactive isotopes tritium, i.e., ³H, and carbon-14, i.e., ¹⁴C, are particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence are preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described in the Preparations and Examples as set out below using an appropriate isotopically-labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compound of the invention. As used herein, the term “solvate” refers to an aggregate that comprises one or more molecules of a compound of the invention with one or more molecules of solvent. In some embodiments, the solvent is water, in which case the solvate is a hydrate. Alternatively, in other embodiments, the solvent is an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. In some aspects, the compound of the invention is a true solvate, while in other cases, the compound of the invention merely retains adventitious water or is a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents, or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The compounds of the invention, or their pharmaceutically acceptable salts, contain one or more asymmetric centers and may thus give rise to enantiomers, diastereoisomers, and other stereoisomeric forms that are defined, in terms of absolute stereochemistry, as (R)- or (S)- or, as (D)- or (L)- for amino acids. The present invention is meant to include all such possible isomers, as well as their racemic, scalemic, and optically pure forms. Optically active (+) and (−), (R)- and (S)-, or (D)- and (L)-isomers may be prepared using chiral synthons, chiral catalysts, or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The present invention includes all manner of rotamers and conformationally restricted states of a compound of the invention. Atropisomers, which are stereoisomers arising because of hindered rotation about a single bond, where energy differences due to steric strain or other contributors create a barrier to rotation that is high enough to allow for isolation of individual conformers, are also included.

A “stereoisomer” refers to a compound made up of the same atoms bonded by the same bonds but having different three-dimensional structures, which are not superimposable. The present invention contemplates various stereoisomers and mixtures thereof and includes “enantiomers”, which refers to two stereoisomers whose molecules are nonsuperimposeable mirror images of one another. For example, the carbon and phosphorous atoms marked with an “*” in the following structure are stereocenters. All stereoisomers of the compounds disclosed herein are also included in the scope of the invention.

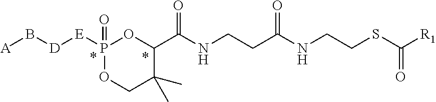

The various substituents (e.g., R₁, D, B, A) also include stereocenters in some embodiments and all such stereocenters and stereoisomeric mixtures are included in the scope of the present invention.

The present invention includes tautomers of any of the disclosed compounds.

Additional definitions are set forth throughout this disclosure.

Compounds

In certain aspects, the present invention provides compounds having the formula (I):

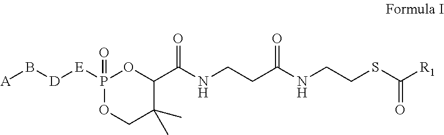

or a pharmaceutically acceptable salt thereof, wherein:

E is O or NR₂;

D is absent, C₁-C₃ alkylene, C(O)O(alkylene) or aryl, wherein each of said C₁-C₃ alkylene, C(O)O(alkylene) and aryl is unsubstituted or substituted with R₃;

B is absent, C₁-C₃ alkylene, C₃-C₆ cycloalkylene, (C₁-C₃ alkylene)NR₂, C(O)NR₂(alkylene), aryl, heteroaryl or heterocyclyl, wherein each of said C₁-C₃ alkylene, C₃-C₆ cycloalkylene, C(O)NR₂(alkylene), aryl, heteroaryl and heterocyclyl is unsubstituted or substituted with R₆;

A is absent, H, OR₅, R₅C(O), R₅OC(O), R₅OC(O)O, R₅C(O)O, R₅C(O)S, NR₂R₅C(O), NR₂R₅C(O)O, R₅C(O)NR₂, R₅C(O)ONR₂, R₅S(O)NR₂, R₅SO₂NR₂,NR₂R₅, C₁-C₆ alkyl, C₃-C₆ cycloalkyl, heterocyclyl, aryl or heteroaryl, wherein each of said C₁-C₆ alkyl, C₃-C₆ cycloalkyl, heterocyclyl, aryl and heteroaryl is unsubstituted or substituted with R₆;

R₁ is H, C₁-C₆ alkyl, aryl, arylalkyl, heteroaryl, heteroarylalkyl, heterocyclyl, heterocyclylalkyl, C₃-C₆ cycloalkyl, or cycloalkylalkyl, wherein each of said C₁-C₆ alkyl, aryl, arylalkyl, heteroaryl, heteroarylalkyl, heterocyclyl, heterocyclylalkyl, C₃-C₆ cycloalkyl, and cycloalkylalkyl is unsubstituted or substituted with R₆;

R₂ is H or C₁-C₆ alkyl;

R₃ is H, C₁-C₆ alkyl, hydroxy, amino, arylalkyl, heteroarylalkyl or C₃-C₆ cycloalkyl, wherein each of said C₁-C₆ alkyl, arylalkyl, heteroarylalkyl and C₃-C₆ cycloalkyl is unsubstituted or substituted with R₄;

R₄ is C₁-C₆ alkyl, C₁-C₆ alkoxy, hydroxy or amino;

R₅ is H, C₁-C₆ alkyl, C₁-C₆ alkoxy, aryl, arylalkyl, heteroaryl, heteroarylalkyl, heterocyclyl, heterocyclylalkyl, C₃-C₆ cycloalkyl, cycloalkylalkyl, amino, alkylamino, dialkylamino, aminoalkyl, alkylaminoalkyl or dialkylaminoalkyl, wherein each of said C₁-C₆ alkyl, C₁-C₆ alkoxy, aryl, arylalkyl, heteroaryl, heteroarylalkyl, heterocyclyl, heterocyclylalkyl, amino, alkylamino, dialkylamino, aminoalkyl, alkylaminoalkyl and dialkylaminoalkyl is unsubstituted or substituted with R₆;

R₆ is C₁-C₆ alkyl, C₁-C₆ alkoxy, hydroxyl, amino, halo, oxo, CN, NO₂, SF₅, heterocyclyl, heterocyclylalkyl, aryl, arylalkyl, C₃-C₆ cycloalkyl, C₃-C₄ spiro-substituted cycloalkyl, cycloalkylalkyl, SO₂R₇, R₇C(O), R₇C(O)NR₂ or C(O)OR₈, wherein each of said C₁-C₆ alkyl, C₁-C₆ alkoxy, heterocyclyl, heterocyclylalkyl, aryl, arylalkyl, C₃-C₆ cycloalkyl, C₃-C₄ spiro-substituted cycloalkyl and cycloalkylalkyl is unsubstituted or substituted with R₇;

R₇ is C₁-C₆ alkyl, C₁-C₆ alkoxy, hydroxyl, halo, oxo, CN, NO₂, SF₅, amino, alkylamino or dialkylamino; and

R₈ is H, C₁-C₆ alkyl or arylalkyl; or

D is absent, and A, B, and E together form a 6-membered heterocyclic or heteroaryl ring, wherein said heterocyclic or heteroaryl ring is unsubstituted or substituted with R₆.

In some embodiments, a compound of Formula I of the present invention has (R)-absolute stereochemistry at the carbon atom marked with an “*” in the following structure:

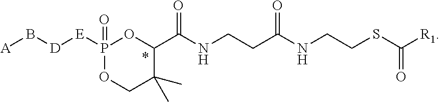

In further embodiments of the compound of Formula I, E is O.

In some embodiments, E is O and D is absent, C₁-C₃ alkylene or C(O)O(alkylene). In certain embodiments, E is O and D is C₁-C₃ alkylene or C(O)O(alkylene).

In some embodiments, E is O and D is C₁-C₃ alkylene. In some embodiments wherein E is O, D is methylene.

In some embodiments, E is O and D is C(O)O(alkylene). In some embodiments wherein E is O, D is C(O)OCH₂.

In some embodiments, E is O and D is absent.

In further embodiments of the compound of Formula I, B is absent, (C₁-C₃ alkylene)NR₂ or (C₁-C₃ alkylene)NR₂ substituted with R₆.

In some embodiments, E is O and B is absent.

In still other embodiments, E is O and B is (C₁-C₃ alkylene)NR₂. In certain embodiments, E is O; B is (C₁-C₃ alkylene)NR₂; and R₂ is Hydrogen.

In some embodiments, E is O and B is (C₁-C₃ alkylene)NR₂ substituted with R₆. In some embodiments, E is O; B is (C₁-C₃ alkylene)NR₂ substituted with R₆, R₂ is Hydrogen; and R₆ is C₁-C₆ or C(O)OR₈. In certain embodiments, E is O; B is absent, (C₁-C₃ alkylene)NR₂ or (C₁-C₃ alkylene)NR₂ substituted with R₆; and R₂ is Hydrogen and R₆ is methyl. In certain embodiments, E is O; B is absent, (C₁-C₃ alkylene)NR₂ or (C₁-C₃ alkylene)NR₂ substituted with R₆, R₂ is Hydrogen; R₆ is C(O)OR₈; and R₈ is Hydrogen, methyl or arylalkyl.

In further embodiments of the compound of Formula I, A is R₅OC(O), R₅OC(O)O, R₅C(O)O, R₅C(O)S, aryl or aryl substituted with R₆.

In some embodiments of the compound of Formula I, E is O and A is R₅OC(O), R₅OC(O)O, R₅C(O)O, R₅C(O)S, aryl or aryl substituted with R₆. In some embodiments, E is O and A is R₅C(O)O, R₅C(O)S, or aryl. In some embodiments, E is O and A is R₅C(O)O or R₅C(O)S.

In some embodiments, E is O and A is R₅C(O)O. In certain embodiments, E is O; A is R₅C(O)O; and R₅ is C₁-C₆ alkyl, C₁-C₆ alkyl substituted with R₆, aryl, aryl substituted with R₆, heteroryl or heteroaryl substituted with R₆. In certain embodiments, E is O; A is R₅C(O)O; and R₅ is C₁-C₆ alkyl. In certain embodiments, E is O; A is R₅C(O)O; and R₅ is C₁-C₆ alkyl substituted with R₆, aryl substituted with R₆ or heteroaryl substituted with R₆. In certain embodiments, E is O; A is R₅C(O)O; R₅ is C₁-C₆ alkyl substituted with R₆, aryl substituted with R₆ or heteroaryl substituted with R₆; and R₆ is C₁-C₆ alkyl, C₁-C₆ alkyl substituted with R₇, C₁-C₆ alkoxy, amino or halo. In certain embodiments, E is O; A is R₅C(O)O; R₅ is C₁-C₆ alkyl substituted with R₆, aryl substituted with R₆ or heteroaryl substituted with R₆; and R₆ is C₁-C₆ alkyl. In certain embodiments, E is O; A is R₅C(O)O; R₅ is C₁-C₆ alkyl substituted with R₆, aryl substituted with R₆ or heteroaryl substituted with R₆; R₆ is C₁-C₆ alkyl substituted with R₇; and R₇ is halo.

In some embodiments, E is O; A is R₅C(O)S; and R₅ is C₁-C₆ alkyl, C₁-C₆ alkyl substituted with R₆, aryl, aryl substituted with R₆, heteroryl or heteroaryl substituted with R₆. In certain embodiments, E is O; A is R₅C(O)S; and R₅ is C₁-C₆ alkyl. In certain embodiments, E is O; A is R₅C(O)S; and R₅ is C₁-C₆ alkyl substituted with R₆, aryl substituted with R₆ or heteroaryl substituted with R₆. In certain embodiments, E is O; A is R₅C(O)S; R₅ is C₁-C₆ alkyl substituted with R₆, aryl substituted with R₆ or heteroaryl substituted with R₆; and R₆ is C₁-C₆ alkyl, C₁-C₆ alkyl substituted with R₇, C₁-C₆ alkoxy, amino or halo. In certain embodiments, E is O; A is R₅C(O)S; R₅ is C₁-C₆ alkyl substituted with R₆, aryl substituted with R₆ or heteroaryl substituted with R₆; and R₆ is C₁-C₆ alkyl. In certain embodiments, E is O; A is R₅C(O)S; R₅ is C₁-C₆ alkyl substituted with R₆, aryl substituted with R₆ or heteroaryl substituted with R₆; R₆ is C₁-C₆ alkyl substituted with R₇; and R₇ is halo.

In still further embodiments, a compound of Formula I is provided, wherein: E is O; D is C₁-C₃ alkylene or C(O)O(alkylene); B is absent, (C₁-C₃ alkylene)NR₂ or (C₁-C₃ alkylene)NR₂ substituted with R₆; A is OR₅, R₅C(O)O, R₅C(O)S or aryl; R₁ is C₁-C₆ alkyl; R₂ is H; R₅ is C₁-C₆ alkyl, C₁-C₆ alkyl substituted with R₆, aryl substituted with R₆ or heteroaryl substituted with R₆; R₆ is C₁-C₆ alkyl, C₁-C₆ alkyl substituted with R₇, C₁-C₆ alkoxy, amino or halo; and R₇ is halo.

In still further embodiments, a compound of Formula I is provided, wherein: E is O; D is C₁-C₃ alkylene; B is absent; A is R₅C(O)O; R₁ is C₁-C₆ alkyl; R₅ is C₁-C₆ alkyl.

In still further embodiments, a compound of Formula I is provided, wherein: E is O; D is C₁-C₃ alkylene; B is absent; A is R₅C(O)O; R₁ is C₁-C₆ alkyl; R₅ is aryl substituted with R₆; and R₆ is C₁-C₆ alkyl.

In still further embodiments, a compound of Formula I is provided, wherein: E is O; D is C₁-C₃ alkylene; B is absent; A is R₅C(O)O; R₁ is C₁-C₆ alkyl; R₅ is aryl substituted with R₆; and R₆ is C₁-C₆ alkyl.

In further embodiments, a compound of Formula I is provided, wherein: E is O; D is C₁-C₃ alkylene; B is absent; A is R₅C(O)O; R₁ is C₁-C₆ alkyl; R₅ is heteroaryl substituted with R₆; and R₆ is alkyl.

In still further embodiments of the compound of Formula I, E is NR₂. In some embodiments, E is NR₂ and R₂ is Hydrogen.

In some embodiments, E is NR₂; and D is C₁-C₃ alkylene or C₁-C₃ alkylene substituted with R₃. In some embodiments, E is NR₂; R₂ is Hydrogen; and D is C₁-C₃ alkylene or C₁-C₃ alkylene substituted with R₃.

In some embodiments, E is NR₂; and D is methylene substituted with R₃. In some embodiments, E is NR₂; R₂ is Hydrogen; and D is methylene substituted with R₃.

In some embodiments, E is NR₂; D is C₁-C₃ alkylene or C₁-C₃ alkylene substituted with R₃; and R₃ is C₁-C₆ alkyl. In some embodiments, E is NR₂; R₂ is Hydrogen; D is C₁-C₃ alkylene or C₁-C₃ alkylene substituted with R₃; and R₃ is C₁-C₆ alkyl.

In some embodiments, E is NR₂; D is methylene substituted with R₃; and R₃ is C₁-C₆ alkyl. In some embodiments, E is NR₂; R₂ is Hydrogen; D is methylene substituted with R₃; and R₃ is C₁-C₆ alkyl.

In still further embodiments of the compound of Formula I, E is NR₂; and B is absent, heterocyclyl or heterocyclyl substituted with R₆. In some embodiments, E is NR₂; R₂ is Hydrogen; and B is absent, heterocyclyl or heterocyclyl substituted with R₆.

In some embodiments of the compound of Formula I, E is NR₂; and B is absent. In some embodiments, E is NR₂; R₂ is Hydrogen; and B is absent.

In still further embodiments of the compound of Formula I, E is NR₂; and A is OR₅, R₅C(O), R₅C(O)S, R₅OC(O), R₅C(O)O, NR₂R₅C(O), R₅C(O)NR₂, R₅S(O)NR₂, R₅SO₂NR₂, NR₂R₅, C₁-C₆ alkyl, C₃-C₆ cycloalkyl, heterocyclyl, aryl or heteroaryl. In some embodiments, E is NR₂; R₂ is Hydrogen; and A is OR₅, R₅C(O), R₅C(O)S, R₅OC(O), R₅C(O)O, NR₂R₅C(O), R₅C(O)NR₂, R₅S(O)NR₂, R₅SO₂NR₂, NR₂R₅, C₁-C₆ alkyl, C₃-C₆ cycloalkyl, heterocyclyl, aryl or heteroaryl.

In some embodiments of the compound of Formula I, E is NR₂; and A is OR₅, R₅C(O), R₅C(O)S, R₅OC(O) or R₅C(O)O. In some embodiments, E is NR₂; R₂ is Hydrogen; and A is OR₅, R₅C(O), R₅C(O)S, R₅OC(O) or R₅C(O)O.

In some embodiments of the compound of Formula I, E is NR₂; and A is R₅OC(O), R₅C(O)S or R₅C(O)O. In some embodiments, E is NR₂; R₂ is Hydrogen; and A is R₅OC(O), R₅C(O)S or R₅C(O)O.

In some embodiments of the compound of Formula I, E is NR₂; and A is R₅OC(O). In some embodiments, E is NR₂; R₂ is Hydrogen; and A is R₅OC(O).

In some embodiments of the compound of Formula I wherein E is NR₂ and A is OR₅, R₅C(O), R₅C(O)S, R₅OC(O), R₅C(O)O, NR₂R₅C(O), R₅C(O)NR₂, R₅S(O)NR₂, R₅SO₂NR₂, or NR₂R₅, R₅ is C₁-C₆ alkyl or arylalkyl. In some embodiments of the compound of Formula I wherein E is NR₂ and A is OR₅, R₅C(O), R₅C(O)S, R₅OC(O), R₅C(O)O, NR₂R₅C(O), R₅C(O)NR₂, R₅S(O)NR₂, R₅SO₂NR₂, or NR₂R₅, R₅ is C₁-C₆ alkyl.

In still further embodiments, a compound of Formula I is provided, wherein: E is NR₂; D is C₁-C₃ alkylene or C₁-C₃ alkylene substituted with R₃; B is absent; A is OR₅, R₅C(O), R₅C(O)S, R₅OC(O) or R₅C(O)O; R₁ is C₁-C₆ alkyl; R₂ is H; R₃ is C₁-C₆ alkyl; and R₅ is C₁-C₆ alkyl or arylkyl.

In still further embodiments, a compound of Formula I is provided, wherein: E is NR₂; D is C₁-C₃ alkylene or C₁-C₃ alkylene substituted with R₃; B is absent; A is OR₅, R₅C(O), R₅C(O)S, R₅OC(O) or R₅C(O)O; R₁ is C₁-C₆ alkyl; R₂ is H; R₃ is C₁-C₆ alkyl; and R₅ is methyl.

In still further embodiments, a compound of Formula I is provided, wherein: E is NR₂; D is C₁-C₃ alkylene substituted with R₃; B is absent; A is R₅OC(O); R₁ is C₁-C₆ alkyl; R₂ is H; R₃ is C₁-C₆ alkyl; and R₅ is C₁-C₆ alkyl.

In still further embodiments, a compound of Formula I is provided, wherein: E is NR₂; D is C₁-C₃ alkylene substituted with R₃; B is absent; A is R₅OC(O); R₁ is C₁-C₆ alkyl; R₂ is H; R₃ is C₁-C₆ alkyl; and R₅ is arylkyl.

In another embodiment, in any of the aforementioned compounds, R₁ may be C₁-C₆ alkyl. In particular embodiments, R₁ is methyl.

In various different embodiments, the compound has one of the structures set forth in Table 1 below.

TABLE 1
Exemplary compounds.

| Cmpnd No. | Structure | Chemical Name |
|---|---|---|
| R1001 | 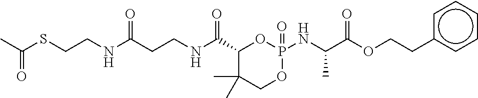 | phenethyl ((4R)-4-((3-((2- (acetylthio)ethyl)amino)-3- oxopropyl)carbamoyl)-5,5- dimethyl-2-oxido-1,3,2- dioxaphosphinan-2-yl)-L- alaninate |
| R1002 | 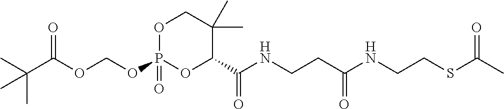 [SMILES: CC(=O)SCCNC(=O)CCNC(=O)[C@@H]1O[P@@](=O)(OCOC(=O)C(C)(C)C)OCC1(C)C] | (((2S,4R)-4-((3-((2- (acetylthio)ethyl)amino)-3- oxopropyl)carbamoyl)-5,5- dimethyl-2-oxido-1,3,2- dioxaphosphinan-2- yl)oxy)methyl pivalate |
| R1003 | 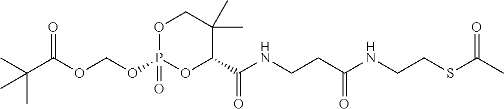 [SMILES: CC(=O)SCCNC(=O)CCNC(=O)[C@@H]1O[P@](=O)(OCOC(=O)C(C)(C)C)OCC1(C)C] | (((2R,4R)-4-((3-((2- (acetylthio)ethyl)amino)-3- oxopropyl)carbamoyl)-5,5- dimethyl-2-oxido-1,3,2- dioxaphosphinan-2- yl)oxy)methyl pivalate |
| R1004 | 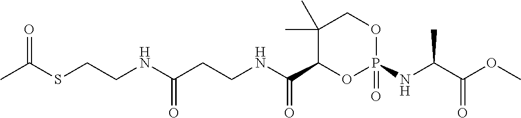 [SMILES: COC(=O)[C@H](C)N[P@]1(=O)OCC(C)(C)[C@H](C(=O)NCCC(=O)NCCSC(C)=O)O1] | methyl ((2S,4R)-4-((3-((2- (acetylthio)ethyl)amino)-3- oxopropyl)carbamoyl)-5,5- dimethyl-2-oxido-1,3,2- dioxaphosphinan-2-yl)-L- alaninate |
| R1005 | 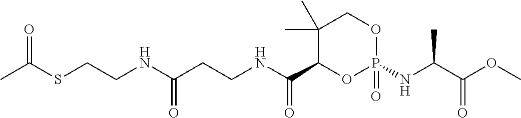 [SMILES: COC(=O)[C@H](C)N[P@@]1(=O)OCC(C)(C)[C@H](C(=O)NCCC(=O)NCCSC(C)=O)O1] | methyl ((2R,4R)-4-((3-((2- (acetylthio)ethyl)amino)-3- oxopropyl)carbamoyl)-5,5- dimethyl-2-oxido-1,3,2- dioxaphosphinan-2-yl)-L- alaninate |
| R1006 | 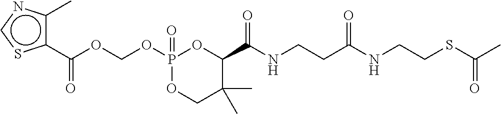 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 4- methylthiazole-5-carboxylate |
| R1007 | 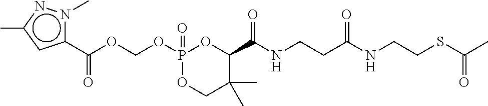 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 1,3-dimethyl- |
| | | 1H-pyrazole-5-carboxylate |
| R1008 | 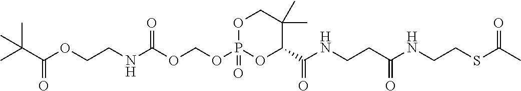 [SMILES: CC(=O)SCCNC(=O)CCNC(=O)[C@@H]1OP(=O)(OCOC(=O)NCCOC(=O)C(C)(C)C)OCC1(C)C] | 2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl pivalate |
| R1009 | 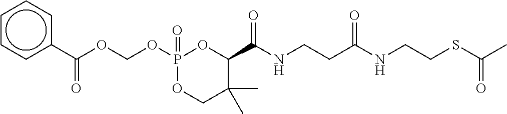 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl benzoate |
| R1010 | 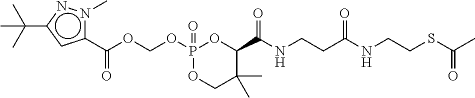 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3-(tert-butyl)- |
| | | 1-methyl-1H-pyrazole-5- |
| | | carboxylate |
| R1011 | 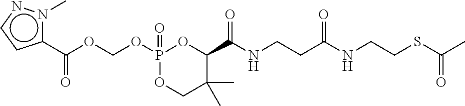 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 1-methyl-1H- |
| | | pyrazole-5 -carboxylate |
| R1012 | 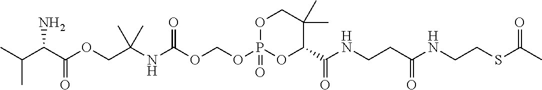 [SMILES: CC(=O)SCCNC(=O)CCNC(=O)[C@@H]1OP(=O)(OCOC(=O)NC(C)(C)COC(=O)[C@@H](N)C(C)C)OCC1(C)C] | 2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)-2-methylpropyl |
| | | L-valinate |
| R1013 | 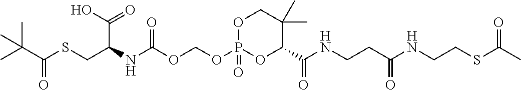 [SMILES: CC(=O)SCCNC(=O)CCNC(=O)[C@@H]1OP(=O)(OCOC(=O)N[C@@H](CSC(=O)C(C)(C)C)C(=O)O)OCC1(C)C] | N-(((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl)- |
| | | S-pivaloyl-L-cysteine |
| R1014 | 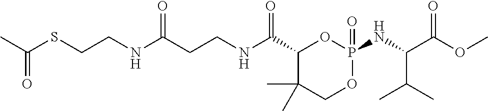 [SMILES: COC(=O)[C@@H](N[P@@]1(=O)OCC(C)(C)[C@H](C(=O)NCCC(=O)NCCSC(C)=O)O1)C(C)C] | methyl ((2R,4R)-4-((3-((2- (acetylthio)ethyl)amino)-3- oxopropyl)carbamoyl)-5,5- dimethyl-2-oxido-1,3,2- dioxaphosphinan-2-yl)- L-valinate |
| R1015 | 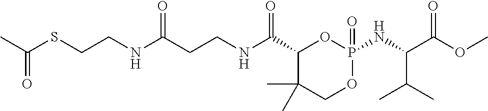 [SMILES: COC(=O)[C@@H](N[P@]1(=O)OCC(C)(C)[C@H](C(=O)NCCC(=O)NCCSC(C)=O)O1)C(C)C] | methyl ((2S,4R)-4-((3-((2- (acetylthio)ethyl)amino)-3- oxopropyl)carbamoyl)-5,5- dimethyl-2-oxido-1,3,2- dioxaphosphinan-2-yl)-L- valinate |
| R1016 | 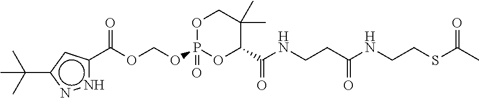 | (((2S,4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3-(tert-butyl)- |
| | | 1H-pyrazole-5-carboxylate |
| R1017 | 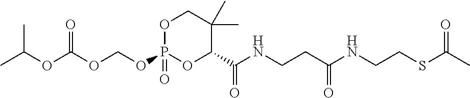 [SMILES: CC(=O)SCCNC(=O)CCNC(=O)[C@@H]1O[P@@](=O)(OCOC(=O)OC(C)C)OCC1(C)C] | S-(2-(3-((2S,4R)-2- (((isopropoxycarbonyl) oxy)methoxy)-5,5- dimethyl-2-oxido-1,3,2- dioxaphosphinane-4- carboxamido)propanamido) |
| | | ethyl) ethanethioate |
| R1018 | 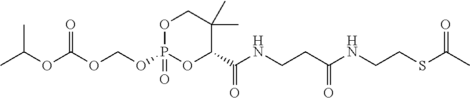 [SMILES: CC(=O)SCCNC(=O)CCNC(=O)[C@@H]1O[P@](=O)(OCOC(=O)OC(C)C)OCC1(C)C] | S-(2-(3-((2R,4R)-2- (((isopropoxycarbonyl)oxy) methoxy)-5,5-dimethyl-2- oxido-1,3,2- dioxaphosphinane-4- carboxamido)propanamido) |
| | | ethyl) ethanethioate |
| R1019 | 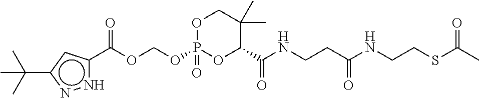 | (((2R,4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3-(tert-butyl)- |
| | | 1H-pyrazole-5-carboxylate |
| R1020 | 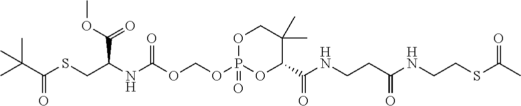 [SMILES: COC(=O)[C@H](CSC(=O)C(C)(C)C)NC(=O)OCOP1(=O)OCC(C)(C)[C@H](C(=O)NCCC(=O)NCCSC(C)=O)O1] | methyl N-(((((4R)-4-((3-((2- (acetylthio)ethyl)amino)-3- oxopropyl)carbamoyl)-5,5- dimethyl-2-oxido-1,3,2- dioxaphosphinan-2- yl)oxy)methoxy)carbonyl)- |
| | | S-pivaloyl-L-cysteinate |
| R1021 | 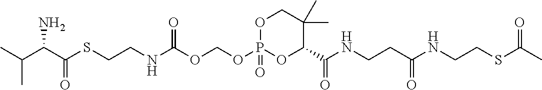 [SMILES: CC(=O)SCCNC(=O)CCNC(=O)[C@@H]1OP(=O)(OCOC(=O)NCCSC(=O)[C@@H](N)C(C)C)OCC1(C)C] | S-(2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl) (2S)-2-amino- |
| | | 3-methylbutanethioate |
| R1022 | 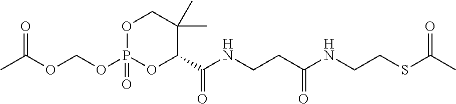 [SMILES: CC(=O)OCOP1(=O)OCC(C)(C)[C@H](C(=O)NCCC(=O)NCCSC(C)=O)O1] | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl acetate |
| R1023 | 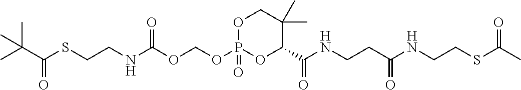 [SMILES: CC(=O)SCCNC(=O)CCNC(=O)[C@@H]1OP(=O)(OCOC(=O)NCCSC(=O)C(C)(C)C)OCC1(C)C] | S-(2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl) 2,2- |
| | | dimethylpropanethioate |
| R1024 | 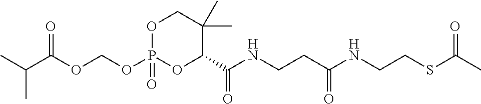 [SMILES: CC(=O)SCCNC(=O)CCNC(=O)[C@@H]1OP(=O)(OCOC(=O)C(C)C)OCC1(C)C] | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl isobutyrate |
| R1025 | 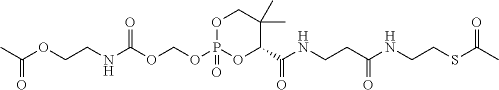 [SMILES: CC(=O)OCCNC(=O)OCOP1(=O)OCC(C)(C)[C@H](C(=O)NCCC(=O)NCCSC(C)=O)O1] | 2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl acetate |
| R1026 | 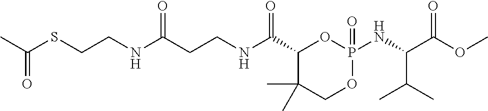 [SMILES: COC(=O)[C@@H](NP1(=O)OCC(C)(C)[C@H](C(=O)NCCC(=O)NCCSC(C)=O)O1)C(C)C] | methyl ((4R)-4-((3-((2- (acetylthio)ethyl)amino)-3- oxopropyl)carbamoyl)-5,5- dimethyl-2-oxido-1,3,2- dioxaphosphinan-2-yl)-L- valinate |
| R1027 | 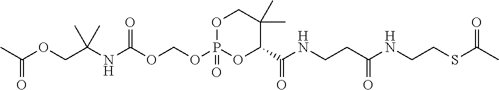 [SMILES: CC(=O)OCC(C)(C)NC(=O)OCOP1(=O)OCC(C)(C)[C@H](C(=O)NCCC(=O)NCCSC(C)=O)O1] | 2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)-2-methylpropyl |
| | | acetate |
| R1028 | 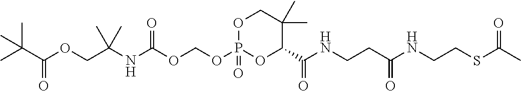 [SMILES: CC(=O)SCCNC(=O)CCNC(=O)[C@@H]1OP(=O)(OCOC(=O)NC(C)(C)COC(=O)C(C)(C)C)OCC1(C)C] | 2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)-2-methylpropyl |
| | | pivalate |
| R1029 | 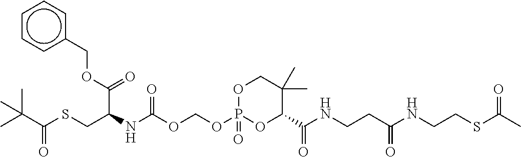 | benzyl N-(((((4R)-4-((3-((2- (acetylthio)ethyl)amino)-3- oxopropyl)carbamoyl)-5,5- dimethyl-2-oxido-1,3,2- dioxaphosphinan-2- yl)oxy)methoxy)carbonyl)- S-pivaloyl-L-cysteinate |
| R1030 | 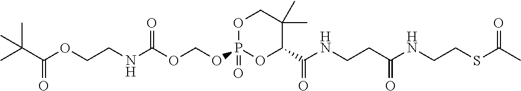 [SMILES: CC(=O)SCCNC(=O)CCNC(=O)[C@@H]1O[P@@](=O)(OCOC(=O)NCCOC(=O)C(C)(C)C)OCC1(C)C] | 2-((((((2S,4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl pivalate |
| R1031 | 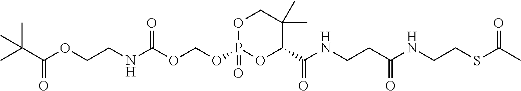 [SMILES: CC(=O)SCCNC(=O)CCNC(=O)[C@@H]1O[P@](=O)(OCOC(=O)NCCOC(=O)C(C)(C)C)OCC1(C)C] | 2-((((((2R,4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl pivalate |
| R1032 | 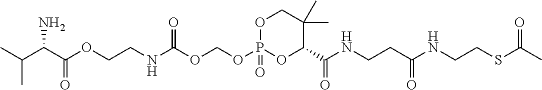 [SMILES: CC(=O)SCCNC(=O)CCNC(=O)[C@@H]1OP(=O)(OCOC(=O)NCCOC(=O)[C@@H](N)C(C)C)OCC1(C)C] | 2-((((((4R)-4-((3-((2- (acetylthio)ethyl)amino)-3- oxopropyl)carbamoyl)-5,5- dimethyl-2-oxido-1,3,2- dioxaphosphinan-2- |
| | | yl)oxy)methoxy)carbonyl) |
| | | amino)ethyl L-valinate |
| R1033 | 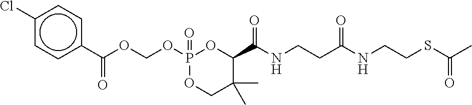 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 4- chlorobenzoate |
| R1034 | 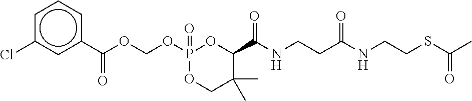 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3- chlorobenzoate |
| R1035 | 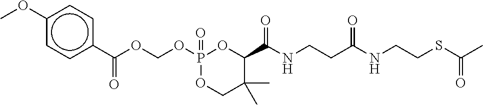 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 4- methoxybenzoate |
| R1036 | 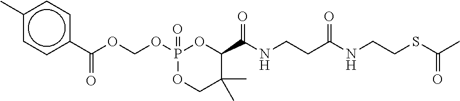 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 4- methylbenzoate |
| R1037 | 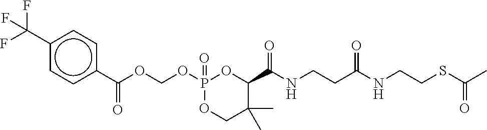 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 4- (trifluoromethyl)benzoate |
| R1038 | 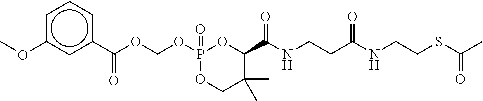 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3- |
| | | methoxybenzoate |
| R1039 | 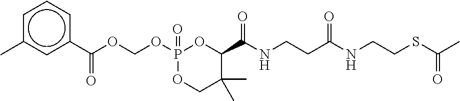 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3- |
| | | methylbenzoate |
| R1040 | 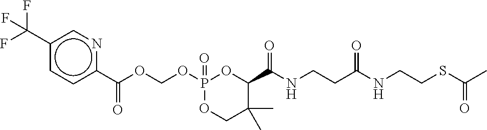 | (((4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 5- (trifluoromethyl)picolinate |
| R1041 | 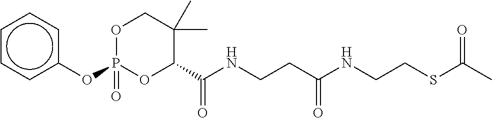 | S-(2-(3-((2S,4R)-5,5- dimethyl-2-oxido-2-phenoxy- 1,3,2-dioxaphosphinane-4- carboxamido)propanamido) ethyl) ethanethioate |
| R1042 | 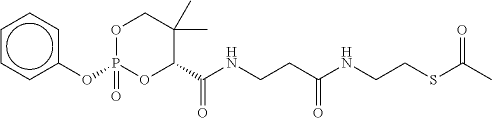 | S-(2-(3-((2R,4R)-5,5- dimethyl-2-oxido-2-phenoxy- 1,3,2-dioxaphosphinane-4- carboxamido)propanamido) ethyl) ethanethioate |
| R1043 | 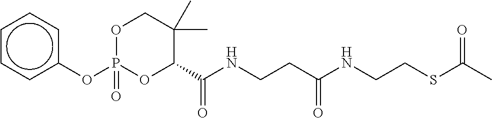 | S-(2-(3-((4R)-5,5-dimethyl- 2-oxido-2-phenoxy-1,3,2- dioxaphosphinane-4- carboxamido)propanamido) ethyl) ethanethioate |
| R1044 | 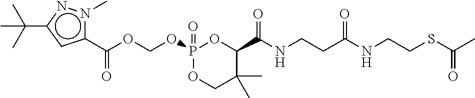 | (((2R,4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3-(tert-butyl)- |
| | | 1-methyl-1H-pyrazole-5- |
| | | carboxylate |
| R1045 | 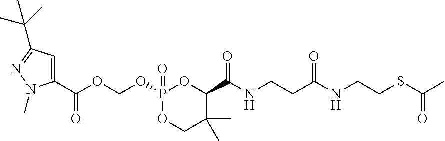 [SMILES: CC(=O)SCCNC(=O)CCNC(=O)[C@@H]1O[P@@](=O)(OCOC(=O)c2cc(C(C)(C)C)nn2C)OCC1(C)C] | (((2S,4R)-4-((3-((2- (acetylthio)ethyl)amino)-3- oxopropyl)carbamoyl)-5,5- dimethyl-2-oxido-1,3,2- dioxaphosphinan-2- yl)oxy)methyl 3-(tert-butyl)- 1-methyl-1H-pyrazole-5- carboxylate |
| R1046 | 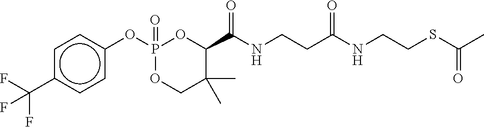 | S-(2-(3-((4R)-5,5-dimethyl- 2-oxido-2-(4- (trifluoromethyl)phenoxy)- 1,3,2-dioxaphosphinane-4- carboxamido)propanamido) ethyl) ethanethioate |
| R1047 | 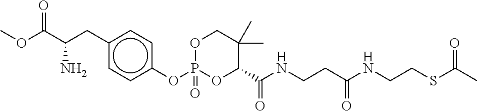 | methyl (2S)-3-(4-(((4R)-4-((3- ((2-(acetylthio)ethyl)amino)- 3-oxopropyl)carbamoyl)-5,5- dimethyl-2-oxido-1,3,2- dioxaphosphinan-2- yl)oxy)phenyl)-2- aminopropanoate |

Synthesis of Compounds

Yet another embodiment of the invention is a method of preparing a compound of Formula I.

### Synthesis of Cyclic Phosphates by Method A

In one approach, cyclic phosphate compounds of the invention can be prepared by Method A below. Briefly, a pantothenate ester is treated with phosphorousoxychloride followed by benzyl alcohol to provide a cyclic phosphate. The benzyl group is then removed with hydrogen gas and palladium on carbon. The resulting phosphodiester is then treated with a chloromethyl ester. Removal of the pantothenate ester and coupling to an S-acyl 2-aminoethanethiol provides the desired compound.

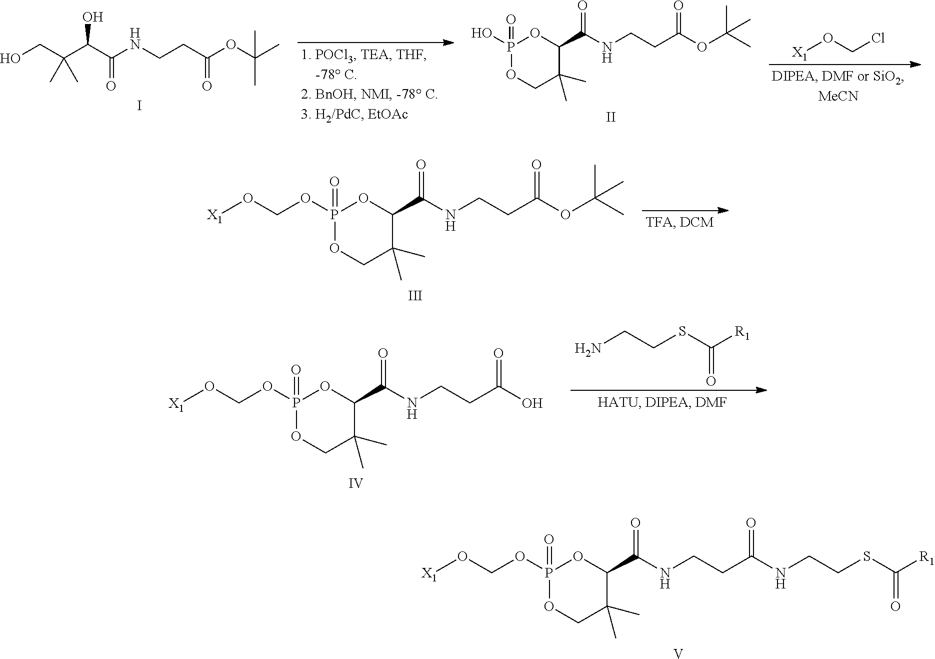

### Synthesis of Cyclic Phosphoramidates by Method B

In another approach, cyclic phosphoramidates can be prepared as outlined in Method B below. Briefly, pantothenic acid can be coupled to an S-acyl 2-aminoethanethiol under standard conditions. In a separate reaction phenoxyphosphoryldichloride is treated with an amine. The resulting phosphoramidochloridate is then coupled to the diol, compound VI, and finally cyclized under basic conditions.

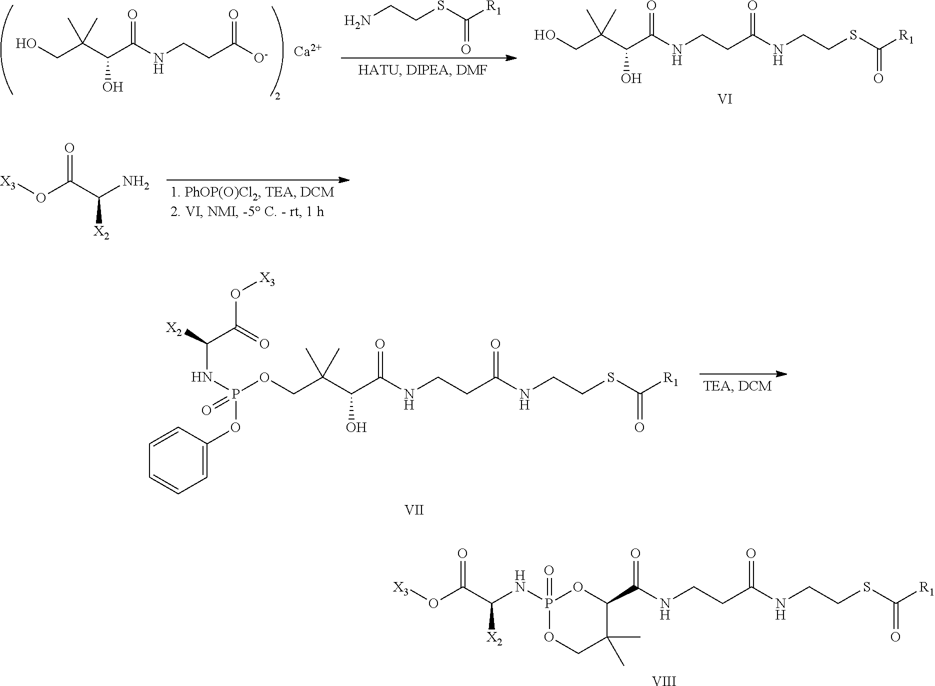

### Synthesis of Cyclic Phosphates by Method C

In another approach, cyclic phosphates can be prepared as outlined in Method C below. Briefly, compound VI, constructed as described above, is treated with a phosphoryldichloro ester to provide the desired cyclic phosphates.

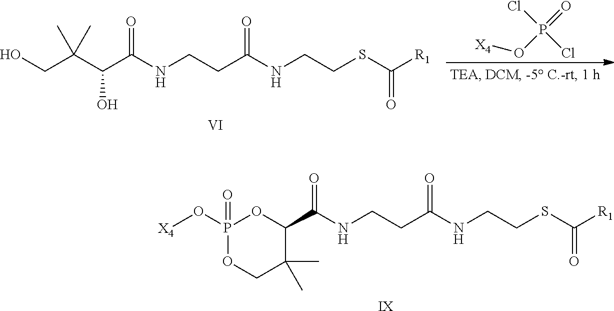

Pharmaceutical Compositions and Methods of Treatment

In certain aspects, the present invention provides pharmaceutical compositions comprising a compound of the present invention, and a pharmaceutically acceptable excipient. In one embodiment, the pharmaceutical composition includes an effective amount of the compound to treat a neurologic disorder. In some embodiments, a pharmaceutical composition comprising a compound having a structure as set forth in Table 1 and a pharmaceutically acceptable excipient is provided. The pharmaceutical compositions may be a dosage unit form, such as a tablet, capsule, liquid, suspension, or sachet.

Yet another aspect is a method of increasing Coenzyme A production in a subject in need thereof by administering to the subject an effective amount of a compound or pharmaceutical composition of the present invention. In one embodiment, the subject in need of increased Coenzyme A production exhibits overexpression of an enzyme for which Coenzyme A is a substrate. In one embodiment, the subject in need of increased Coenzyme A production has a deficiency of Coenzyme A production, a deficiency of pantothenate kinase enzyme, and/or a deficiency of 4′-phosphopantetheine or 4′-phosphopantothenic acid. In one embodiment, the subject in need thereof has a defect or mutation in a pantothenate kinase gene (PANK). In one embodiment, a method of increasing Coenzyme A production in a subject having a defect in the PANK1, PANK2, PANK3, or PANK4 gene, or any combination thereof, is provided. In one embodiment, a method of increasing Coenzyme A production in a subject having a defect in the PANK2 gene is provided. In one embodiment, the compound administered to increase Coenzyme A production has a structure as set forth in Table 1.

Yet another embodiment is a method of treating a subject having a disorder associated with pantothenate kinase enzyme deficiency comprising administering to a subject in need thereof an effective amount of a compound or pharmaceutical composition of the present invention. In one embodiment, the compound administered to treat a subject having a disorder associated with pantothenate kinase enzyme deficiency has a structure as set forth in Table 1. In one embodiment, the disorder is pantothenate kinase-associated neurodegeneration (PKAN). In one embodiment, the disorder is 4′-phosphopantothenic acid deficiency. In another embodiment, the subject exhibits neurodegeneration with brain iron accumulation. In one embodiment, the subject having a disorder associated with pantothenate kinase enzyme deficiency has a pantothenate kinase gene (PANK) defect. In one embodiment, a method of treating a subject having a disorder associated with pantothenate kinase enzyme deficiency is provided, wherein the subject has a defect in the PANK1, PANK2, PANK3, or PANK4 gene, or any combination thereof. In one embodiment, a method of treating a subject having a disorder associated with pantothenate kinase enzyme deficiency is provided, wherein the subject has a PANK1 gene defect. In one embodiment, a method of treating a subject having a disorder associated with pantothenate kinase enzyme deficiency is provided, wherein the subject has a PANK2 gene defect. In one embodiment, a method of treating a subject having a disorder associated with pantothenate kinase enzyme deficiency is provided, wherein the subject has a PANK3 gene defect. In one embodiment, a method of treating a subject having a disorder associated with pantothenate kinase enzyme deficiency is provided, wherein the subject has a PANK4 gene defect.

Yet another embodiment is a method of treating a subject having a disorder associated with Coenzyme A deficiency, comprising administering to the subject an effective amount of a compound or pharmaceutical composition of the present invention. In one embodiment, the compound administered to treat a subject having a disorder associated with Coenzyme A deficiency has a structure as set forth in Table 1.

Yet another embodiment is a method of treating a condition associated with abnormal neuronal function in a subject, comprising administering to the subject an effective amount of a compound or pharmaceutical composition of the present invention. In one embodiment, the condition may be Parkinson's disease, dystonia, extrapyramidal effects, dysphagia, rigidity and/or stiffness of limbs, choreoathetosis, tremor, dementia, spasticity, muscle weakness, or seizure. In one embodiment, the compound administered to treat the condition associated with abnormal neuronal function has a structure as set forth in Table 1.

Yet another embodiment is a method of treating a condition associated with neuronal cell iron accumulation in a subject in need thereof, comprising administering to the subject an effective amount of a compound or pharmaceutical composition of the present invention. In one such embodiment, the compound administered to treat the condition associated with neuronal cell iron accumulation has a structure as set forth in Table 1.

Another embodiment is a method of treating a subject having neurodegeneration with brain iron accumulation, comprising administering to the subject an effective amount of a compound or pharmaceutical composition of the present invention. In one embodiment, the compound administered to treat a subject having neurodegeneration with brain iron accumulation has a structure as set forth in Table 1. In one embodiment, the subject having neurodegeneration with brain iron accumulation has pantothenate kinase-associated neurodegeneration (PKAN).

Another embodiment is a method of treating a subject having a disorder associated with deficiency of 4′-phosphopantothenoylcysteine synthase, comprising administering to a subject in need thereof an effective amount of a compound or pharmaceutical composition of the present invention. In one embodiment, the compound administered to treat a subject with deficiency of 4′-phosphopantothenoylcysteine synthase has a structure as set forth in Table 1.

Another embodiment is a method of treating a subject having a disorder associated with deficiency of 4′-phosphopantothenoylcysteine decarboxylase, comprising administering to a subject in need thereof an effective amount of a compound or pharmaceutical composition of the present invention. In one embodiment, the compound administered to treat a subject with deficiency of 4′-phosphopantothenoylcysteine decarboxylase has a structure as set forth in Table 1.

In any of the aforementioned embodiments, the subject being treated or in need thereof may be a child. In some embodiments, the child is 10 to 15 years old. In other embodiments, the subject being treated or in need thereof is an adult.

Pharmaceutical Formulations and Routes of Administration

The compounds and pharmaceutical compositions of the present invention may be administered by a variety of routes, including orally, nasally, buccally, sublingually, and by injection (e.g., subcutaneously, intravenously, intrathecally, and intraperitoneally).

The compounds or pharmaceutical compositions may be administered orally in the form of a solid or liquid dosage form. In both, the compounds or pharmaceutical compositions may be coated in a material to protect it from the action of acids and other natural conditions which may inactivate the compound. The compounds or pharmaceutical compositions may be formulated as aqueous solutions, liquid dispersions, (ingestible) tablets, buccal tablets, troches, capsules, elixirs, suspensions, syrups, and wafers. The oral dosage forms may include excipients known in the art, such as binders, disintegrating agents, flavorants, antioxidants, and preservatives. Liquid dosage forms may include diluents such as saline or an aqueous buffer.

For nasal administration, the preparation can contain a compound or pharmaceutical composition of the invention, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens. Solutions or suspensions may be applied directly to the nasal cavity by conventional means, for example with a dropper, pipette, or spray. The formulations may be provided in single or multidose form. In the case of a dropper or pipette, this may be achieved by the patient administering an appropriate predetermined volume of the solution or suspension. In the case of a spray, this may be achieved for example by means of a metering atomizing spray pump. To improve nasal delivery and retention, the compounds according to the invention may be encapsulated with cyclodextrins, or formulated with their agents expected to enhance delivery and retention in the nasal mucosa.

The compounds and pharmaceutical compositions may also be administered by injection. Formulations suitable for injection may include sterile aqueous solutions (where water soluble) or dispersions, and sterile powders for the extemporaneous preparation of sterile injectable solutions or dispersions. The composition may be sterile and be fluid to the extent that easy syringability exists. It may be stable under the conditions of manufacture and storage and be preserved against the contaminating action of microorganisms such as bacteria and fungi. The carrier can be a solvent or dispersion medium containing, for example, water, ethanol, polyol (such as glycerol, propylene glycol, and liquid polyethylene glycol), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, and ascorbic acid. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound or pharmaceutical composition in the required amount in an appropriate solvent with one or a combination of ingredients enumerated above, as required, followed by sterile filtration. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying, which yields a powder of the active ingredient (i.e., the therapeutic compound) plus any additional desired ingredient from a previously sterile-filtered solution thereof.

The actual dosage amount of the compound administered to a subject may be determined by physical and physiological factors such as age, sex, body weight, severity of condition, the type of disease being treated, previous or concurrent therapeutic interventions, idiopathy of the subject, and the route of administration. These factors may be determined by a skilled artisan. The practitioner responsible for administration will typically determine the concentration of active ingredient(s) in a composition and appropriate dose(s) for the individual subject.

In one embodiment, a human subject is administered daily doses of from about 0.01 mg/kg to about 100 mg/kg.

Single or multiple doses of the compound or pharmaceutical composition are contemplated. Desired time intervals for delivery of multiple doses can be determined by one of ordinary skill in the art employing no more than routine experimentation. As an example, subjects may be administered two doses daily at approximately 12 hour intervals. In some embodiments, the compound or pharmaceutical composition is administered once a day. In other embodiments, the compound or pharmaceutical composition is delivered two times a day. In still other embodiments, the compound or pharmaceutical composition is delivered three times a day.

The compounds or pharmaceutical compositions may be administered on a routine schedule. As used herein a routine schedule refers to a predetermined designated period of time. The routine schedule may encompass periods of time which are identical or which differ in length, as long as the schedule is predetermined. For instance, the routine schedule may involve administration four times a day, three times a day, twice a day, every day, every two days, every three days, every four days, every five days, every six days, a weekly basis, a monthly basis or any set number of days or weeks there-between. In some embodiments, the predetermined routine schedule may involve administration on a twice daily basis for the first week, followed by a daily basis for several months. In some embodiments, the predetermined routine schedule may involve administration on a twice daily basis for the first week, followed by a daily basis thereafter. In some embodiments, the predetermined routine schedule may involve administration three times a day for a specified period, followed by administration two times a day or one time a day for several months. For example, in some embodiments, the compound or pharmaceutical composition is administered three times a day for a period of one to four weeks, followed by administration two times a day or one time a day for a period of greater than or equal to 12 weeks.

In some embodiments, the invention provides that the compound or pharmaceutical composition may be taken orally and that the timing of which is or is not dependent upon food intake. Thus, for example, the compound or pharmaceutical composition can be taken every morning and/or every evening, regardless of when the subject has eaten or will eat.

Combination Therapy

In addition to being used as a monotherapy, the compounds and pharmaceutical compositions may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

The additional agent or agents may be selected from any agent or agents useful for treating a neurological disorder, for example any agent or agents useful for treating a deficiency of pantothenate kinase, 4′-phosphopantothenic acid, 4′-phosphopantetheine, or Coenzyme A. In one embodiment, the additional agent or agent is useful in improving cognitive function, e.g., an acetylcholinesterase inhibitor, such as physostigmine, neostigmine, pyridostigmine, ambenonium, demarcarium, rivastigmine, galantamine, donezepil, and combinations thereof. In another embodiment, the additional agent or agents is an iron chelator, such as deferiprone, deferoxamine, deferasirox, and combinations thereof.

### EXAMPLES

Unless otherwise stated, all reagents used in the Examples were obtained from commercial sources and were used as received without further purification. The NMR spectrometers utilized were Bruker instruments operating at the indicated frequencies. UPLC-MS analysis was conducted on a Waters UPLC system with both Diode Array detection and Electrospray (+′ve and −′ve ion) MS detection. The stationary phase was a Waters Acquity UPLC BEH C18 1.7 um 2.1×50 mm column. The mobile phase was H₂O containing 0.1% formic acid (A) and MeCN containing 0.1% formic acid (B) in the following linear gradient: 90% A (0.1 min), 90%-0% A (2.5 min), 0% A (0.3 min), 90% A (0.1 min) with a flow rate of 0.5 mL/min. Reverse phase (C18) column chromatography was carried out using as mobile phase H₂O containing 0.1% of TFA and MeCN containing 0.1% of TFA.

### Example 1

### (((2S,4R)-4-((3-((2-(acetylthio)ethyl)amino)-3-oxopropyl)carbamoyl)-5,5-dimethyl-2-oxido-1,3,2-dioxaphosphinan-2-yl)oxy)methyl pivalate (Compound No. R1002)

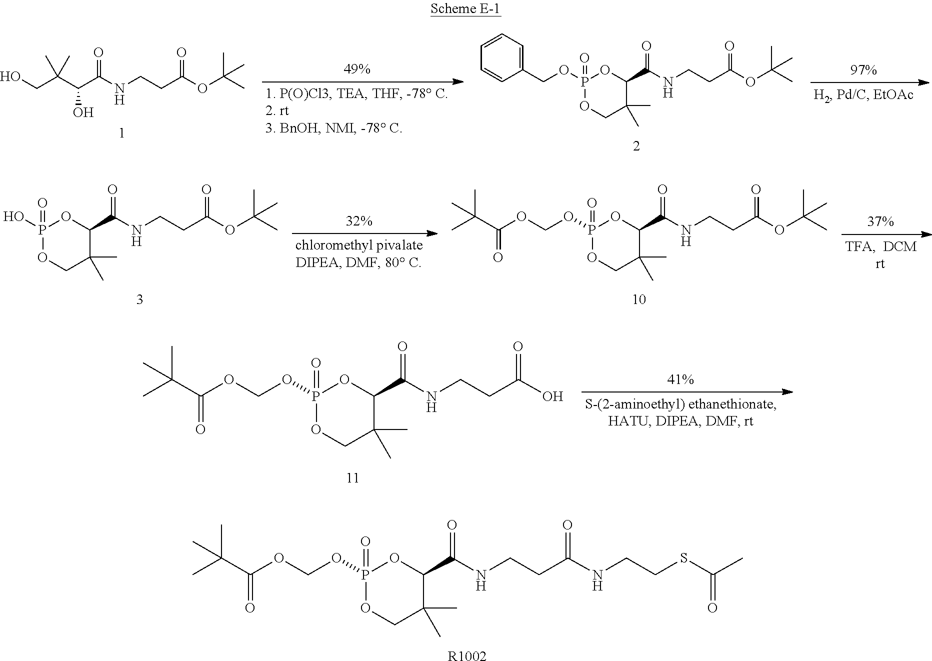

(((2S,4R)-4-((3-((2-(acetylthio)ethyl)amino)-3-oxopropyl)carbamoyl)-5,5-dimethyl-2-oxido-1,3,2-dioxaphosphinan-2-yl)oxy)methyl pivalate (Compound No. R1002) was synthesized via the method shown above.

### Step 1. tert-butyl 3-((4R)-2-hydroxy-5,5-dimethyl-2-oxido-1,3,2-dioxaphosphinane-4-carboxamido) propanoate (Compound 3, Scheme E-1)

tert-butyl(R)-3-(2,4-dihydroxy-3,3-dimethylbutanamido)propanoate (compound 1, Scheme E-1 above) (1.0 eq) was dissolved in THF (0.3 M) and sequentially a solution of POCl₃ in THF (1.0 eq, 6 M) and TEA in THF (1.1 eq, 2.6 M) were added dropwise at −78° C. Stirring was continued at this temperature for 0.5 h then the cooling bath was removed and the reaction mixture was warmed to ambient temperature over 1 h. The mixture was cooled again to −78° C. then treated sequentially with a solution of benzyl alcohol in THF (1.2 eq, 7M) and 1-methylimidazole in THF (2.1, 7M). Stirring was continued at −78° C. for 0.5 h then the mixture was allowed to warm slowly to ambient temperature and after 12 h was quenched with H₂O. The organic solvent was evaporated and DCM was added. The organic layer was separated and washed sequentially with 5% aqueous citric acid solution, water, and saturated aqueous NaCl, and then dried over Na₂SO₄. Filtration and solvent removal afforded a residue that was purified by flash chromatography column on SiO₂ eluting with PE/EtOAc to afford a mixture of two diastereoisomers 59:41* of tert-butyl 3-((4R)-2-(benzyloxy)-5,5-dimethyl-2-oxido-1,3,2-dioxaphosphinane-4-carboxamido)propanoate (compound 2, Scheme E-1 above) (49%) as a white solid. ¹H-NMR (400 MHz, CDCl₃, 300 K) δ 7.47-7.39 (m, 5H), 6.91* and 6.81 (bs, 1H), 5.26-5.24 and 5.16-5.13* (d, 2H, =9.8 Hz and J=9.4 Hz*), 4.77 and 4.42* (m, 1H), 4.39-4.35 and 4.064.03* (m, 1H), 4.17-4.12 and 3.86-3.73* (m, 1H), 3.60-3.53 (m, 1H), 3.51-3.53 (m, 1H), 2.51-2.42 (m, 2H), 1.43* and 1.45 (s, 9H), 1.19 and 1.11* (s, 3H), 1.10 and 1.09* (s, 3H). ³¹P-NMR (162 MHz, CDCl₃, 300K) δ −4.59, −8.67*. UPLC tR 1.72*, 1.80 min; MS (ES+) m/z 428 [M+H]⁺.

Tert-butyl 3-((4R)-2-(benzyloxy)-5,5-dimethyl-2-oxido-1,3,2-dioxaphosphinane-4-carboxamido) propanoate (compound 2, Scheme E-1 above) (1.0 eq) was dissolved in EtOAc (0.1 M) and then treated with Pd/C (10% w/w). The mixture was stirred for 2 h at room temperature under an atmosphere of hydrogen gas. The reaction was judged complete by UPLC analysis and was purged with N₂ (g). The catalyst was removed by filtration and the filtrate was evaporated to afford the title compound (97%) as a colorless oil. ¹H-NMR (400 MHz, DMSO-d₆, 300 K) δ 7.93 (bs, 1H), 4.42 (s, 1H), 4.01 (d, 1H, J=11.8), 3.76 (dd, 1H, J_{AB}=10.8, J_{HP}=23.7 Hz), 3.35-3.26 (m, 2H), 2.36 (t, 2H, J=7.0 Hz), 1.39 (s, 9H), 0.98 (s, 3H), 0.94 (s, 3H). ³¹P-NMR (162 MHz, DMSO-d₆, 300 K) 6-8.03. UPLC tR 0.90 min; MS (ES+) m/z 338 [M+H]⁺.

### Step 2. (((4R)-4-((3-(tert-butoxy)-3-oxopropyl)carbamoyl)-5,5-dimethyl-2-oxido-1,3,2-dioxaphosphinan-2-yl)oxy)methyl pivalate (Compound 10, Scheme E-1 above)

A solution of tert-butyl 3-((4R)-2-hydroxy-5,5-dimethyl-2-oxido-1,3,2-dioxaphosphinane-4-carboxamido)propanoate (compound 3, Scheme E-1 above) (1.0 eq) in DMF (0.21M) was cooled to −78° C. and treated with chloromethyl pivalate (1.7 eq) and N,N-diisopropylethylamine (2.8 eq). The cooling bath was removed and the mixture was warmed to room temperature over 1 h before heating at 80° C. for 12 h. The mixture was cooled and washed sequentially with aqueous HCl (1 N), saturated aqueous solution of NaHCO₃ and saturated aqueous NaCl. After removal of the solvent under vacuum a residue was obtained that was purified by flash chromatography column on C18 eluting with H₂O/MeCN. Fractions containing product were concentrated under reduced pressure to afford the title compound (32%) as white powder. ¹H-NMR (400 MHz, CDCl₃, 300 K) δ 7.02 (bs, 1H), 5.71 (d, 2H, J_{HP}=13.2 Hz), 4.60 (s, 1H), 4.19 (d, 1H, J=15.6 Hz* and J=11.1 Hz), 3.64-3.56 (m, 1H), 3.52-3.44 (m, 1H), 2.51-2.47 (m, 2H), 1.48 (s, 9H), 1.25 (s, 9H), 1.15 (s, 3H), 1.14 (s, 3H). ³¹P-NMR (162 MHz, CDCl₃, 300 K) 8-9.36. UPLC tR 2.14 min; MS (ES+) m/z 452 [M+H]⁺.

### Step 3. (((2S,4R)-4-((3-((2-(acetylthio)ethyl)amino)-3-oxopropyl)carbamoyl)-5,5-dimethyl-2-oxido-1,3,2-dioxaphosphinan-2-yl)oxy)methyl pivalate (Compound No. R1002)

(((4R)-4-((3-(tert-butoxy)-3-oxopropyl)carbamoyl)-5,5-dimethyl-2-oxido-1,3,2-dioxaphosphinan-2-yl)oxy)methyl pivalate (compound 10, Scheme E-1 above) (1.0 eq) was dissolved in DCM (0.16 M) and TFA (0.37 M) was added dropwise at room temperature. The reaction was stirred for 1 h then the solvent was evaporated and the residue was purified by flash chromatography column C18 eluting with H₂O/CH₃CN. Fractions containing product were concentrated under reduced pressure to give 3-((2S,4R)-5,5-dimethyl-2-oxido-2-((pivaloyloxy)methoxy)-1,3,2-dioxaphosphinane-4-carboxamido)propanoic acid (compound 11, Scheme E-1 above) (37%) as a colorless oil. UPLC tR 1.14 min. MS (ES+) m/z 398 [M+H]⁺.

3-((4R)-5,5-dimethyl-2-oxido-2-((pivaloyloxy)methoxy)-1,3,2-dioxaphosphinane-4-carboxamido)propanoic acid (compound 11, above) (1.0 eq) was dissolved in DMF (0.2 M) and HATU (1.5 eq) followed by S-(2-aminoethyl) ethanethioate (1.5 eq) and DIPEA (2.0 eq) were added. The mixture was stirred for 45 min at room temperature and, after removal of the solvent in vacuo, dissolved in DCM. The organic phase was washed sequentially with aqueous NaOH (1N), water and saturated aqueous NaCl. The organic layer was dried over Na₂SO₄, filtered and evaporated to recover a residue that was purified by flash chromatography column on SiO₂ eluting with PE/EtOAc to afford the title compound (41%) as an orange solid. ¹H-NMR (400 MHz, CDCl₃, 300 K) δ 6.94 (bt, 1H), 5.90 (bt, 1H), 5.67-5.58 (m, 2H), 4.48 (s, 1H), 4.10-4.07 (d, 1H, J=11.9 Hz), 3.81-3.71 (m, 1H), 3.56-3.45 (m, 2H), 3.40-3.36 (q, 2H, J=4.2 Hz), 2.97-2.94 (t, 2H, J=7.8 Hz), 2.35-2.32 (t, 2H, J=7.8 Hz), 2.30 (s, 3H), 1.16 (s, 9H), 1.06 (s, 3H), 1.05 (s, 3H). ³¹P-NMR (162 MHz, CDCl₃, 300 K) 8-10.71. UPLC tR 1.40 min. MS (ES+) m/z 497 [M+H]⁺.

### Example 2

### 2-((((((2R,4R)-4-((3-((2-(acetylthio)ethyl)amino)-3-oxopropyl)carbamoyl)-5,5-dimethyl-2-oxido-1,3,2-dioxaphosphinan-2-yl)oxy)methoxy)carbonyl)amino)ethyl pivalate (Compound No. R1031)

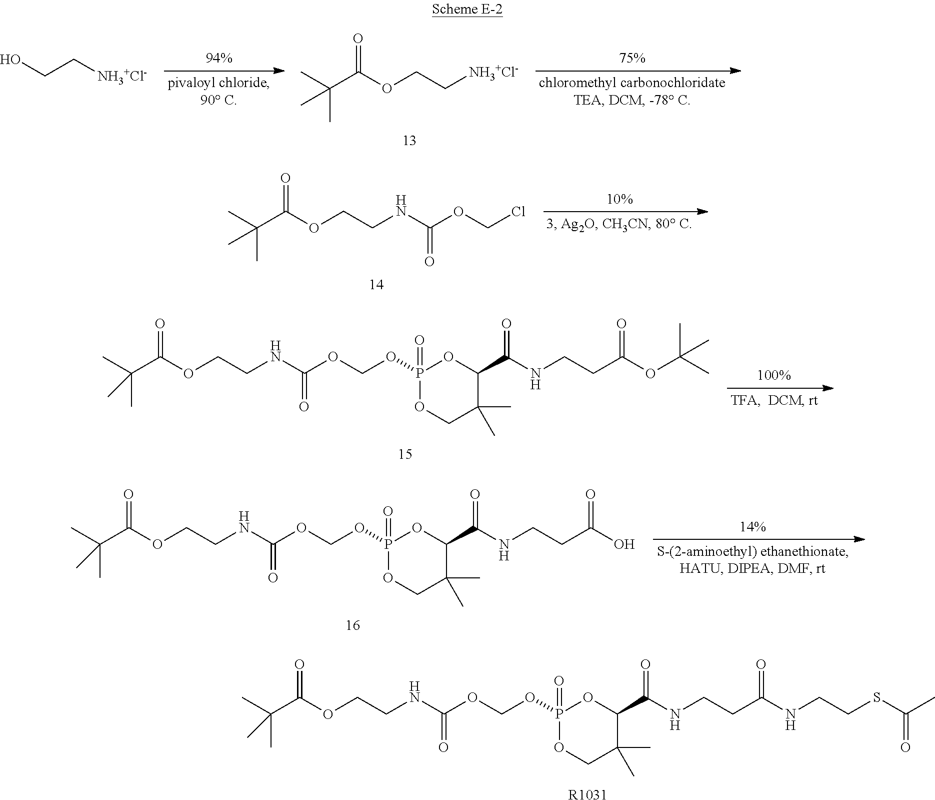

### Step 1. 2-(pivaloyloxy)ethan-1-aminium chloride (Compound 13, Scheme E-2 above)

To 2-hydroxyethan-1-aminium chloride (1.0 eq), pivaloyl chloride (4.0 eq) was added at room temperature. Stirring was continued at 90° C. for 4 h and at room temperature for 15 h. The solid formed was filtered and washed with diethyl ether to afford the title compound (94%) as a white solid. ¹H-NMR (400 MHz, DMSO-d₆, 300 K) δ 8.27 (bs, 3H), 4.19-4.17 (t, 2H, J=4.0 Hz), 3.08-3.06 (t, 2H, J=4.0 Hz), 1.18 (s, 9H).

### Step 2. 2-(((chloromethoxy)carbonyl)amino)ethyl pivalate (Compound 14, Scheme E-2 above)

To a solution of 2-(pivaloyloxy)ethan-1-aminium chloride (1.0 eq) (compound 13, Scheme E-2 above) in DCM (0.2 M), a solution of chloromethyl carbonochloridate (1.1 eq) in DCM (0.2 M) followed by TEA (2.0 eq) were added at −78° C. The reaction mixture was warmed to room temperature over 75 min and filtered on solka floc. The organic phase was washed with water and saturated aqueous NaCl, and then dried over Na₂SO4. After filtration and solvent removal under vacuum the title compound (75%) was obtained as a colorless oil. ¹H-NMR (400 MHz, CDCl₃, 300 K) δ 5.77 (s, 2H), δ 5.12 (bs, 1H), 4.21-4.18 (t, 2H, J=4.0 Hz), 3.55-3.52 (t, 2H, J=4.0 Hz), 1.23 (s, 9H).

### Step 3. 2-((((((2R,4R)-4-((3-(tert-butoxy)-3-oxopropyl)carbamoyl)-5,5-dimethyl-2-oxido-1,3,2-dioxaphosphinan-2-yl)oxy)methoxy)carbonyl)amino)ethyl pivalate (Compound 15, Scheme E-2)

A solution of tert-butyl 3-((4R)-2-hydroxy-5,5-dimethyl-2-oxido-1,3,2-dioxaphosphinane-4-carboxamido)propanoate (1.0 eq) (compound 3, see Example 1) and 2-(((chloromethoxy)carbonyl)amino)ethyl pivalate (compound 14, Scheme E-2 above) (1.1 eq) in dry CH₃CN (0.1M) was treated with silver (I) oxide (1.5 eq). Stirring was continued in the dark at 80° C. for 1 h. After cooling to room temperature the mixture was filtered on a plug of solka floc eluting with DCM. The organic solvent was removed to afford a residue that was purified by flash chromatography column on SiO₂ eluting with PE/EtOAc to afford a mixture of diastereoisomers in a 54:46* ratio. The title compound was obtained in a 10% yield. UPLC tR 1.80 min; MS (ES+) m/z 539 [M+H]⁺.

### Step 4. 3-((2R,4R)-5,5-dimethyl-2-oxido-2-((((2-(pivaloyloxy)ethyl)carbamoyl) oxy)methoxy)-1,3,2-dioxaphosphinane-4-carboxamido)propanoic acid (Compound 16, Scheme E-2 above)

2-((((((2R,4R)-4-((3-(tert-butoxy)-3-oxopropyl)carbamoyl)-5,5-dimethyl-2-oxido-1,3,2-dioxaphosphinan-2-yl)oxy)methoxy)carbonyl)amino)ethyl pivalate (compound 15, Scheme E-2 above) (1.0 eq) was dissolved in DCM/TFA (10/1 v/v) (0.1 M) and the reaction mixture was stirred at room temperature for 2 h. The solvent was removed to afford the title compound (100%) that was used directly in the subsequent reaction step. UPLC tR 1.28 min; MS (ES+) m/z 483 [M+H]⁺.

### Step 5. 2-((((((2R,4R)-4-((3-((2-(acetylthio)ethyl)amino)-3-oxopropyl)carbamoyl)-5,5-dimethyl-2-oxido-1,3,2-dioxaphosphinan-2-yl)oxy)methoxy)carbonyl)amino)ethyl pivalate (Compound No. R1031, Scheme E-2 above)

A solution of 3-((2R,4R)-5,5-dimethyl-2-oxido-2-((((2-(pivaloyloxy)ethyl)carbamoyl)oxy)methoxy)-1,3,2-dioxaphosphinane-4-carboxamido)propanoic acid (compound 16, Scheme E-2 above) (1.0 eq) in DMF (0.10M) was treated with HATU (1.5 eq), DIPEA (2.0 eq) and S-(2-aminoethyl) ethanethioate (1.5 eq). The mixture was stirred at room temperature for 10 min. Solvent was then removed under reduced pressure and the residue dissolved in DCM and washed with saturated aqueous solution of NaHCO₃, saturated aqueous NaCl and then dried over Na₂SO₄. After filtration and removal of the solvent a residue was obtained that was purified by preparative RP-HPLC using H₂O/MeCN as eluent. Fractions containing product were concentrated under reduced pressure to afford the title compound (14%). ¹H-NMR (400 MHz, DMSO-d₆, 300 K) δ 8.14-8.11 (t, 1H, J=7.1 Hz), 8.03-8.01 (t, 1H, J=6.9 Hz), 7.81-7.78 (t, 1H, J=7.1 Hz), 5.66-5.59 (m, 2H), 4.70-4.69 (d, 1H, J=4.0 Hz), 4.21-4.17 (m, 1H), 4.10-4.01 (m, 3H), 3.20-3.16 (q, 2H, J=7.1 Hz), 2.91-2.88 (t, 2H, J=6.9 Hz), 2.33 (s, 3H), 2.31-2.28 (m, 2H), 1.13 (s, 9H), 1.04 (s, 3H), 0.98 (s, 3H). ³¹P-NMR (162 MHz, DMSO-d₆, 300 K) δ −7.95. UPLC tR 1.80 min; MS (ES+) m/z 584 [M+H]⁺.

### Example 3

### methyl ((2S,4R)-4-((3-((2-(acetylthio)ethyl)amino)-3-oxopropyl)carbamoyl)-5,5-dimethyl-2-oxido-1,3,2-dioxaphosphinan-2-yl)-L-alaninate (Compound No. R1004)

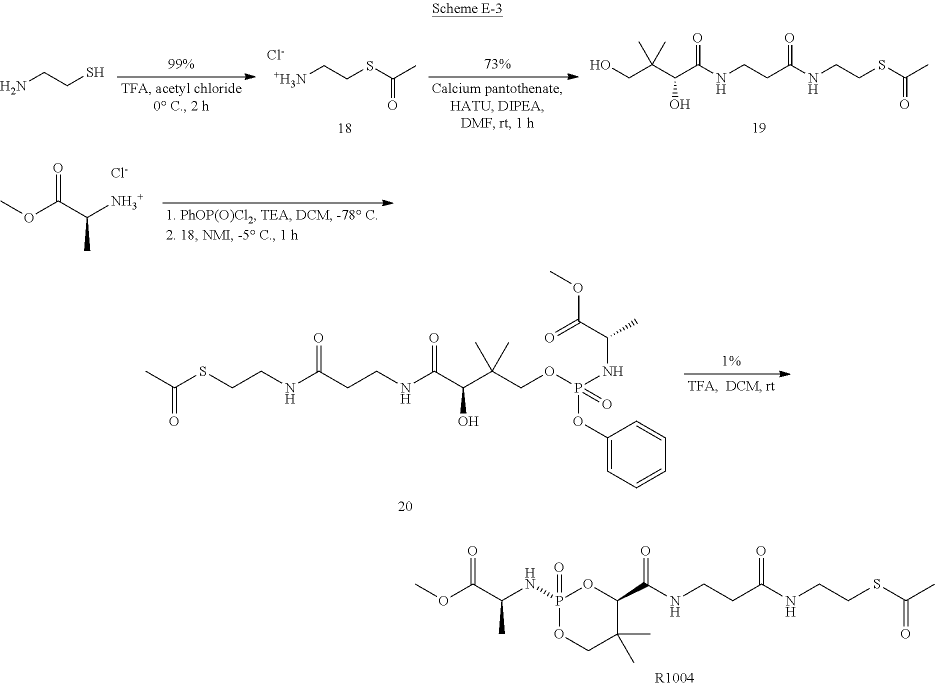

### Step 1. 2-(acetylthio)ethan-1-aminium chloride (Compound 18, Scheme E-3 above)

A solution of 2-aminoethane-1-thiol (1.0 eq) in trifluoroacetic acid (2 M) was cooled at 0° C. and treated with acetyl chloride (1 eq). The reaction mixture was warmed to room temperature and stirred for 2 h. Addition of Et₂O gave, after filtration, the title compound (99%). ¹H-NMR (400 MHz, DMSO-d₆, 300 K) δ 8.32 (bs, 3H), 3.11-3.08 (t, 2H, J=7.8 Hz), 2.92 (m, 2H), 2.36 (s, 3H).

### Step 2. (R)—S-(2-(3-(2,4-dihydroxy-3,3-dimethylbutanamido)propanamido)ethyl) ethanethioate (Compound 19, Scheme E-3 above)

2-(acetylthio)ethan-1-aminium chloride (compound 18, Scheme E-3 above) (1 eq) and HATU (1.1 eq) were added to a solution of calcium ((R)-3-(2,4-dihydroxy-3,3-dimethylbutanamido)propanoate) (0.5 eq) in DMF (0.6M). DIPEA (1.0 eq) was added dropwise and the mixture was stirred at room temperature for 1 h. The organic solvent was evaporated to afford a residue that was purified by flash chromatography column on SiO₂ eluting with DCM/EtOAc/MeOH to afford the title compound (73%). ¹H-NMR (400 MHz, DMSO-d₆, 300 K) δ 8.10-8.08 (bt, 1H), 7.69-7.66 (bt, 1H), 5.36-5.35 (d, 1H, J=4.2 Hz), 4.47-4.44 (bt, 1H), 3.71-3.69 (t, 1H, J=7.8 Hz), 3.32-3.13 (m, 6H), 2.91-2.88 (t, 2H, J=7.8 Hz), 2.33 (s, 3H), 2.27-2.24 (t, 2H, J=8.0 Hz), 0.80 (s, 3H), 0.78 (s, 3H). UPLC tR 0.80 min; MS (ES+) m/z 321 [M+H]⁺.

### Step 3. methyl (((R)-4-((3-((2-(acetylthio)ethyl)amino)-3-oxopropyl)amino)-3-hydroxy-2,2-dimethyl-4-oxobutoxy)(phenoxy)phosphoryl)-L-alaninate (Compound 20, Scheme E-3 above)

The title compound was prepared using the same procedure described in Example 1 (Step 4) and used as crude material.

### Step 4. methyl ((2S,4R)-4-((3-((2-(acetylthio)ethyl)amino)-3-oxopropyl)carbamoyl)-5,5-dimethyl-2-oxido-1,3,2-dioxaphosphinan-2-yl)-L-alaninate (Compound No. R1004)

A solution of methyl R(R)-4-((3-((2-(acetylthio)ethyl)amino)-3-oxopropyl)amino)-3-hydroxy-2,2-dimethyl-4-oxobutoxy)(phenoxy)phosphoryl)-L-alaninate (compound 20, Scheme E-3 above) (1 eq) in DCM (0.20M) was treated with triethylamine (4 eq) at room temperature and the mixture was stirred at this temperature for 24 h. The solvent was evaporated to recover a residue that was purified by preparative RP-HPLC using H₂O/MeCN as eluent to produce, after lyophilization, the title compound (1%) as a colorless oil. ¹H-NMR (400 MHz, DMSO-d₆, 300 K) δ 8.16-8.13 (bt, 1H), 7.87-7.84 (bt, 1H), 6.07-6.02 (dd, 1H, J_{AB}=8.0 Hz, J_{HP}=12.0 Hz), 4.55 (d, 1H, J=2.1 Hz), 4.09-4.05 (dd, 1H, J_{AB}=4.1 Hz, J_{HP}=12.0 Hz), 3.96-3.85 (m, 1H), 3.79-3.71 (dd, 1H, J_{AB}=12.1 Hz, J_{HP}=20.0 Hz), 3.64 (s, 3H), 3.21-3.16 (q, 2H, J=7.8 Hz), 2.92-2.88 (t, 2H, J=7.8 Hz), 2.34 (s, 3H), 2.31-2.28 (m, 2H), 1.32-1.30 (d, 3H, J=8.0 Hz), 0.97 (s, 3H), 0.95 (s, 3H). ³¹P-NMR (162 MHz, DMSO-d₆, 300 K) δ −3.76. UPLC tR 1.06 min. MS (ES+) m/z 468 [M+H]⁺.

### Example 4

### S-(2-(3-((2R,4R)-5,5-dimethyl-2-oxido-2-phenoxy-1,3,2-dioxaphosphinane-4-carboxamido)propanamido)ethyl) ethanethioate (Compound No. 1042) and S-(2-(3-((2S,4R)-5,5-dimethyl-2-oxido-2-phenoxy-1,3,2-dioxaphosphinane-4-carboxamido)propanamido)ethyl) ethanethioate (Compound No. 1041)

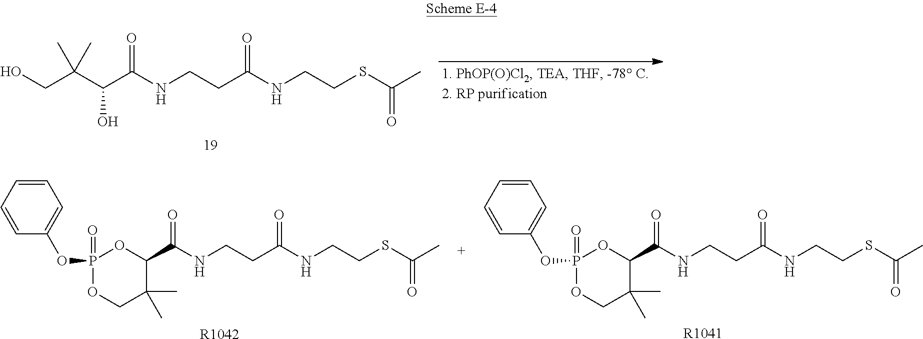

(R)—S-(2-(3-(2,4-dihydroxy-3,3-dimethylbutanamido)propanamido)ethyl) ethanethioate (compound 19, Scheme E-4, above) (1 eq) was dissolved in THF (0.25M) and sequentially a solution of PhOP(O)Cl₂ in THF (1.1 eq, 6 M) and TEA in THF (2.0 eq, 2.6 M) were added dropwise at −78° C. Stirring was continued at this temperature for 0.5 h then the cooling bath was removed and the reaction mixture was warmed to ambient temperature. After 1 h of stirring the reaction was quenched with H₂O. The organic solvent was evaporated and DCM was added. The organic layer was separated and washed sequentially with 5% aqueous citric acid solution, water, and saturated aqueous NaCl, and then dried over Na₂SO₄. Filtration and solvent removal afforded a residue mixture of diastereoisomers that was purified by preparative RP-HPLC using H₂O/MeCN as eluent. Fractions containing the separated diastereoisomers were concentrated under reduced pressure to afford the titled compounds:

Compound No. R1042: yield 2% as colorless oil. ¹H-NMR (400 MHz, DMSO-d₆, 300 K) δ 8.13-8.12 (bs, 2H), 7.44-7.40 (m, 2H), 7.29-7.23 (m, 3H), 4.75 (d, 1H, J=7.3 Hz), 4.32-4.15 (m, 2H), 3.38-3.30 (m, 2H, overlap with H₂O), 3.22-3.17 (q, 2H, J=6.3 Hz), 2.92-2.88 (t, 2H, J=7.0 Hz), 2.33 (s, 3H), 2.30-2.27 (t, 2H, J=6.9 Hz), 1.08 (s, 3H), 0.77 (s, 3H). ³¹P-NMR (162 MHz, DMSO-d₆, 300K) δ −12.93. UPLC tR 1.30 min. MS (ES+) m/z 459 [M+H]⁺.

Compound No. R1041: yield 1.5% as colorless oil. ¹H-NMR (400 MHz, DMSO-d₆, 300 K) δ 8.14-8.09 (bq, 2H), 7.46-7.42 (m, 2H), 7.32-7.25 (m, 3H), 4.85 (s, 1H), 4.34-4.32 (d, 1H, J=10.9 Hz), 4.09-4.00 (dd, 1H, J_{AB}=11.2 Hz, J_{HP}=25.2 Hz), 3.38-3.30 (m, 2H, overlap with H₂O), 3.20-3.15 (q, 2H, J=6.7 Hz), 2.91-2.87 (t, 2H, J=6.7 Hz), 2.33 (s, 3H), 2.31-2.28 (t, 2H, J_{AB}=7.6 Hz), 1.08 (s, 3H), 1.02 (s, 3H). ³¹P-NMR (162 MHz, DMSO-d₆, 300K) δ −14.42. UPLC tR 1.29 min. MS (ES+) m/z 459 [M+H]⁺.

### Example 5

### Exemplary Compounds

Table 2 provides descriptive data, including mass spectrometry data, for some of the compounds shown in Table 1. The compounds in Table 2 were each prepared and analyzed by mass spectrometry and/or ¹H or ³¹P NMR. General methods by which the compounds may be prepared are provided above and indicated in Table 2. Exemplary synthetic procedures are described in more detail in Examples 1-4 above.

TABLE 2
Exemplary compounds.

| Compound | | MS (m/z) | |
|---|---|---|---|
| No. | MWt^{a} | [M + H]⁺ | Synthesis^{b} |
| R1001 | 557.6 | 558 | B |
| R1002 | 496.51 | 497 | A |
| R1004 | 467.47 | 468 | B |
| R1006 | 537.54 | 538 | A |
| R1007 | 534.52 | 535 | A |
| R1008 | 583.59 | 584 | A |
| R1009 | 516.5 | 517 | A |
| R1010 | 576.6 | 577 | A |
| R1011 | 520.49 | 521 | A |
| R1012 | 626.66 | 627 | A |
| R1013 | 643.66 | 644 | A |
| R1014 | 495.53 | 496 | A |
| R1015 | 495.53 | 496 | B |
| R1016 | 562.57 | 563 | A |
| R1017 | 498.48 | 499 | A |
| R1019 | 562.57 | 563 | A |
| R1020 | 657.69 | 658 | A |
| R1021 | 614.67 | 615 | A |
| R1022 | 454.43 | 455 | A |
| R1023 | 599.65 | 600 | A |
| R1024 | 482.49 | 483 | A |
| R1025 | 541.51 | 542 | A |
| R1026 | 495.53 | 496 | B |
| R1027 | 569.56 | 570 | A |
| R1028 | 611.64 | 612 | A |
| R1029 | 733.79 | 734 | A |
| R1030 | 583.59 | 584 | A |
| R1031 | 583.59 | 584 | A |
| R1032 | 598.6 | 599 | A |
| R1033 | 550.95 | 551 | A |
| R1034 | 550.95 | 551 | A |
| R1035 | 546.53 | 547 | A |
| R1036 | 530.53 | 531 | A |
| R1037 | 584.5 | 585 | A |
| R1038 | 546.53 | 547 | A |
| R1039 | 530.53 | 531 | A |
| R1040 | 585.49 | 586 | A |
| R1041 | 458.47 | 459 | C |
| R1042 | 458.47 | 459 | C |
| R1043 | 458.47 | 459 | C |
| R1044 | 576.6 | 578 | A |
| R1045 | 576.6 | 577 | A |
| R1046 | 526.46 | 527 | C |
| R1047 | 559.57 | 560 | C |

^{a}Molecular weight.
^{b}Method of synthesis, as described herein.

### Example 6

### Coenzyme A Assay in PANK2 Silenced Cells

Compounds of the invention show attractive pharmaceutical and biological properties for the treatment of disorders related to decreased Coenzyme A synthesis. Compounds from the invention demonstrate the ability to increase Coenzyme A (CoA or CoA-SH) levels in cell lines (e.g., neuroblastoma) in which the PANK2 gene has been silenced (Table 3).

A human neuroblastoma IMR32 cell line (ATCC) with stably PANK2 silencing was obtained by lentiviral-delivered small hairpin RNA and cultured in MEM (Invitrogen) supplemented with 10% fetal bovine serum, 2 mM glutamine, 1% penicillin-streptomycin, 1 mM sodium pyruvate, 1 mM non-essential amino acids, and 1.5 g/l sodium bicarbonate.

Establishment of a PANK2^{-/-} cell model

For lentiviral shRNA expression, Human Embryonic Kidney HEK-293T cells (ATCC) were transfected with the appropriate pGFP-Lenti-shRNA constructs and packaging plasmids according to manufacturer's protocol (Origene Technologies, Inc.). Four different gene-specific shRNA expression vectors designed against multiple splice variants of PANK2 (Gene ID 80025) were used for transfection. A non-silencing shRNA construct (scrambled shRNA) and an empty vector expressing GFP alone were used as negative controls. The GFP tag subcloned into the lentiviral vectors was used to monitor the transfection efficiency.

IMR32 cells were plated on 150 cm dishes 48 h before transduction with lentiviral particles.

Three days after transduction medium was removed and replaced with fresh medium containing 1 μg/μl puromycin. Medium was replaced every 48 h. The level of PANK2 expression in selected clones was assessed by Western Blot analysis.

### Cell-Based Assay on PANK2^{-/-} Cells

To quantify CoA, PANK2−/− IMR32 cells were plated on 12-well culture plates (Corning) at a density of 0.2×10⁶ cells per well. After 72 h, compounds were freshly dissolved in DMSO and added to the culture medium to yield a final solvent concentration of 0.1% (v/v). Controls with medium containing 0.1% DMSO without test compounds were also included in each plate. Compound treated cells were incubated for 24 h at 37° C. Treatment was repeated after 24 h with newly dissolved compound and cells were further incubated at 37° C. for additional 24 h. Before LC-MS analysis of CoA levels, cells were harvested, counted, and collected in a 15 ml falcon tube and centrifuged at 200×g for 5 min at 4° C. Supernatant was removed and cell pellet was washed in 10 ml of ice-cold PBS. To further confirm that the numbers of cells in each sample were equivalent, an equal fraction of pellet was collected from each sample and subjected to protein determination analysis. After centrifugation and supernatant removal, the cell pellet was rapidly frozen in Liquid Nitrogen and stored at −80° C. until analysis.

Intracellular CoA levels were calculated considering an intracellular volume of 1 million cells=2 μl.

The 1*10⁶ cellular pellet was extracted with 120 μl of aqueous 20% TFA. This solution was stirred for 2 min, sonicated in ultrasonic bath for 2 min, then stirred again for 1 min, and centrifuged for 15 min at 14000 g and at 4° C.

100 μL samples of supernatant were dried under N₂ at 20° C. in the dark.

Samples were re-dissolved in 100 μl of 10 mM NH₄⁺ AcO⁻ buffer pH 5.1+IS (Dextrorphan 50 ng/ml), stirred for 2 min, sonicated in ultrasonic bath for 1 min, then stirred again for 1 min, and injected into LC-MS.

LC-MS/MS was performed using an Agilent HPLC (1100 Series, USA). The LC system was interfaced with an API-4000 Q-Trap triple quadrupole mass spectrometer (AB Sciex, Toronto, Canada) equipped with a TurboIonSpray ionization source operating in positive ion mode. Analyst™ software version 1.6 (AB Sciex, Toronto, Canada) was used for data acquisition and processing. CoA was separated using a Luna C18 column (2.0×50 mm; 5 μm particle size), column at 25° C. and flow rate of 0.2 ml/min. Injection volume was 15 μl. The mobile phases consisted of water containing 10 mM ammonium acetate pH 7 (mobile phase A) and MeCN-2-propanol 9:1 (mobile phase B). Elution was performed using a gradient starting at 2% B, holding at 2% B until 0.1 min, increasing to 98% B at 3.2 min, holding at 98% B until 4.5 min, returning to 2% B at 4.6 min and holding at 2% B until 7.5 min. Precursor ions and MRM transitions used were: CoA m/z 768.1→261.6 and 768.1→136.1.

Results for selected compounds tested in PANK2 silenced cells are reported in Table 3. Results are expressed as fold increase in CoA levels relative to controls (using LC-MS quantification of free CoA).

TABLE 3
Fold increase in Coenzyme A (CoA) levels relative to controls
(PANK2−/− CoA) in PANK2 silenced cells treated with compounds.

| Compound | Concentration | PANK2−/− CoA |
|---|---|---|
| No. | (uM) | Fold increase |
| R1001 | 50 | 3.5 |
| R1002 | 50 | — |
| R1004 | 50 | 2.1 |
| R1006 | 50 | 116.6 |
| R1007 | 50 | 108.7 |
| R1008 | 50 | 41.2 |
| R1009 | 1 | 69.5 |
| R1010 | 1 | 200.0 |
| R1011 | 50 | 101.7 |
| R1012 | 50 | 1.5 |
| R1013 | 50 | 1.0 |
| R1014 | 50 | 1.7 |
| R1015 | 50 | 2.1 |
| R1016 | 1 | 323 |
| R1017 | 1 | 18.4 |
| R1019 | 1 | 28.8 |
| R1020 | 50 | 74.2 |
| R1021 | 50 | 3.7 |
| R1022 | 50 | 114.4 |
| R1023 | 50 | 17.0 |
| R1024 | 1 | 1.4 |
| R1025 | 10 | 1.4 |
| R1026 | 50 | 2.5 |
| R1027 | 50 | 2.8 |
| R1028 | 50 | 5.7 |
| R1029 | 50 | 120 |
| R1030 | 10 | 7.1 |
| R1031 | 50 | 23.2 |
| R1032 | 50 | 1.0 |
| R1040 | 50 | 4.5 |
| R1033 | 1 | 25.8 |
| R1034 | 1 | 29.6 |
| R1035 | 1 | 18.0 |
| R1036 | 1 | 52.2 |
| R1037 | 1 | 30.1 |
| R1038 | 1 | 26.2 |
| R1039 | 1 | 27.4 |
| R1040 | 50 | 4.5 |
| R1041 | 10 | 1.8 |
| R1042 | 10 | 1.0 |
| R1043 | 10 | 1.1 |
| R1044 | 1 | 20.3 |
| R1045 | 1 | 2.3 |
| R1046 | 10 | 1.0 |
| R1047 | 10 | 1.2 |

### Example 7

### Stability of Compounds in Hepatocytes

Stability in hepatocytes for select compounds disclosed in the application was evaluated in two species (mouse and human) according to the following procedure. Compounds and positive control samples were dissolved in 100% DMSO at 5 mM. Cryopreserved hepatocytes were thawed and resuspended in Hepatocyte Basal Medium (HBM-Lonza CC-3199) supplemented with CC-4182 (complete hepatocyte culture medium). Test compounds were diluted into cell suspension (1 million cells/ml) from the stock solutions to have a test compound concentration of 5 μM (0.1% DMSO). Incubation was performed in 24-well plates, at 37° C. in a DUBNOFF water bath, under low shaking. Each compound was tested at 6 time points, in duplicates (0, 15, 30, 60, 120, and 240 min). At each time point, an aliquot of 120 μl was taken and transferred to a 96-well deep plate. The reaction was stopped with the addition of one volume of 100% acetonitrile plus 0.1% formic acid and the appropriate internal standard. Samples then were centrifuged at 1100×g for 30 min at +4° C. and supernatants were transferred to a new 96-deepwell plate. Samples were evaporated under N₂ and reconstituted in H₂O/ACN 0.1% formic Acid (98:2). Analysis was performed without a calibration curve (Acquity UPLC-Waters; Sciex API4000). Time 0 was obtained adding acetonitrile before addition of the test compound. Stability was determined based on analysis of disappearance of the compounds as a function of incubation time. Quantification of test compounds was measured as a peak area relative to an internal standard. The elimination constant, k, is calculated by plotting mean disappearance values on a semi-logarithmic scale and fitting with a best fit linear regression. The half-life (t_{1/2}) expressed in hours was derived using Equation 1:

t_{1/2}=ln 2/(−k). Equation 1:

For those compounds for which half-life could not be calculated, data are reported as: <0.25 or >4. Stability data for compounds in hepatocytes are shown in Table 4.

TABLE 4
Stability as measured by half-life (t_{1/2}) in mouse and human hepatocytes

| | | | t_{1/2} (h) Human | |
|---|---|---|---|---|
| | t_{1/2} (h) Mouse Hepatocytes | | Hepatocytes | |
| Compound | Diastereo- | Diastereo- | Diastereo- | Diastereo- |
| No. | isomer A ^{a} | isomer B ^{b} | isomer A ^{a} | isomer B ^{b} |
| R1002 | <0.25 | NA | <0.25 | NA |
| R1006 | 0.31 | <0.25 | 0.42 | 0.35 |
| R1007 | <0.25 | <0.25 | 0.25 | 0.21 |
| R1008 | <0.25 | <0.25 | 0.29 | <0.25 |

^{a} In all cases, “Diastereoisomer A” refers to the upfield shift in the ³¹P NMR.
^{b} “NA” means not applicable (i.e., only one diastereoisomer tested).

### Example 8

### Stability of Compounds in Plasma and Whole Blood

Compounds disclosed herein have desirable stability properties in plasma and in whole blood.

The stability of selected compounds in human plasma was evaluated according to the following protocol. Compounds and positive control samples were dissolved in 100% DMSO at 3 mM. To investigate the stability of the test compounds in plasma, samples were made by diluting test compounds into plasma from the stock solutions to obtain a test compound concentration of 3 μM (0.1% DMSO). Before addition of a test compound, 990 μl of plasma were preincubated at 37° C. for 5 min in eppendorf. After addition of a test compound, 70 μl for each time point were transferred to a 96-deepwell plate, previously warmed at 37° C. in a DUBNOFF water bath. Each compound was tested at five time points, in duplicate (10, 20, 30, 40 and 60 min). At each time point, an aliquot of 50 μl was taken and transferred to a new 96-deepwell plate, and the reaction was stopped with 200 μl of 100% acetonitrile containing 0.1% formic acid and the appropriate internal standard. Then samples were centrifuged at 1100×g for 30 min at +4° C. and supernatants were transferred to a new 96-deepwell plate. Samples were evaporated under N₂ and reconstituted in H₂O/ACN 0.1% formic Acid (98:2). Analysis was performed without a calibration curve (Acquity UPLC-Waters; SciexAPI4000). Time 0 was obtained by adding acetonitrile before addition of the test compound.

The stability of selected compounds in human whole blood was evaluated according to the following protocol. Compounds and positive control samples were dissolved in 100% DMSO at 3 mM (stock solution). To investigate the stability of the test compounds in whole blood, a 200 μM working solution (WS) in water has been prepared by adding 66.66 μl of each 3 mM stock solution to 933.34 μl of water (6.66% DMSO content). After pre-incubation of the whole blood at 37° C. for 5 min in an eppendorf, 10.5 μl of each working solution were spiked into 689.5 μl of heated blood. The obtained 3 μM solution in blood (0.1% DMSO content) was quickly stirred and 100 μl for each time point were transferred to an eppendorf. Each compound was tested in duplicate at five time points (10, 20, 30, 40 and 60 min). At each time point, the reaction was stopped by quenching the spiked blood (100 μl) with 400 μl of 100% acetonitrile containing 0.1% formic acid. Then samples were centrifuged at 15600 rpm for 15 min at +4° C. and 200 μl of supernatant were transferred to a 96-deepwell plate. Samples were evaporated under N₂ and reconstituted in H₂O/ACN 0.1% formic Acid (98/2 v/v) containing the appropriate internal standard. Analysis was performed without a calibration curve by using an Acquity-UPLC (Waters) coupled to a triple quadrupole mass spectrometer (SciexAPI4000). Time point 0 was obtained by adding 3 μL of each 200 μM working solution to 997 μL of the supernatant obtaining after centrifugation (15600 rpm for 15 min at +4° C.) of the blank matrix quenched with acetonitrile containing 0.1% formic acid.

Stability was determined based on analysis of disappearance of the compounds as a function of incubation time. Quantification of test compounds was measured as a peak area relative to an internal standard. The elimination constant k and half-life (t_{1/2}) were determined as described above in Example 7. For those compounds for which half-life could not be calculated, data are reported as <0.16 or >1. Stability data for compounds in human plasma and whole blood are shown in Tables 5 and 6, respectively.

TABLE 5
Stability as measured by half-life (t_{1/2}) in human plasma

| | t_{1/2} (h) Human Plasma | |
|---|---|---|
| Compound | Diastereoisomer | Diastereoisomer |
| No. | A ^{a} | B ^{b} |
| R1001 | >1 | 0.36 |
| R1002 | 0.72 | NA |

^{a} In all cases, “Diastereoisomer A” refers to the upfield shift in the ³¹P NMR.
^{b} “NA” means not applicable (i.e., only one diastereoisomer tested).

TABLE 6
Stability as measured by half-life (t_{1/2}) in human whole blood

| | t_{1/2} (h) Human Whole Blood | |
|---|---|---|
| Compound | Diastereoisomer | Diastereoisomer |
| No. | A ^{a, b} | B ^{b} |
| R1006 | 0.37 | <0.16 |
| R1007 | 0.34 | <0.16 |
| R1008 | 0.5 | 0.23 |
| R1011 | 0.42 | <0.16 |
| R1012 | 0.41 | NA |
| R1013 | 0.78 | 0.6 |
| R1015 | NA | >1 |
| R1019 | NA | <0.16 |
| R1020 | 0.65 | NA |
| R1021 | 0.32 | NA |
| R1022 | <0.16 | <0.16 |
| R1023 | 0.31 | 0.17 |
| R1031 | 0.41 | 0.25 |

^{a} In all cases, “Diastereoisomer A” refers to the upfield shift in the ³¹P NMR.
^{b} “NA” means not applicable (i.e., only one diastereoisomer tested).

### Example 9

### Permeability in a Porcine Brain Endothelial Cell Model

Compounds disclosed herein show the potential to reach mammalian brain by crossing the blood-brain barrier (BBB) from systemic circulation. Both diastereoisomers of selected exemplary compounds of the invention exhibit permeability in a porcine brain endothelial cell (PBEC) model of the mammalian BBB, as summarized in Table 7. The porcine brain endothelial cell permeability assay is an in vitro BBB model to be used for the prediction of central nervous system (CNS) drug permeability in vivo and for ranking or prioritization of compounds according to their permeability. This system can also be used for mechanistic studies and drug delivery strategies via receptor-mediated transport (transcytosis). The system is a two-dimensional co-culture, non-contact model of two types of primary cells: primary brain endothelial cells obtained from fresh porcine brains, and primary rat astrocytes, obtained from neonatal rats (PBECs/As). This ensures barrier formation and functional expression of key transporters.

The endothelial cells were cultured on rat-tail collagen type I and human fibronectin coated Transwell polycarbonate inserts (surface area 0.7 cm²; pore size 0.4 μm) and the inserts were placed in 24-well plates containing confluent rat astrocytes. This system allows for the formation of a differentiated BBB model suitable for compound permeability in 10 days.

On the day of the experiment, culture medium was removed and cells were pre-incubated for 30 min with HBSS containing 20 mM Hepes pH 7.4 and 0.1% BSA. Donor volume (apical) was 400 μl and receiver volume (basal) 900 μl. Compounds were diluted in assay medium (at the desired concentration) and added to the luminal side (to mimic blood to brain passage). Transport was measured usually after 60 min by detecting the amount of compound from the basal (brain side). The integrity of the cell layers was assessed by measuring the transendothelial electrical resistance (TEER) and by monitoring FITC-dextran (40 KDa) permeation. Inserts with TEER values>500 Ω/cm² were selected for permeability studies. As FITC-dextran cannot freely permeate lipophilic barriers, a high degree of FITC-dextran transport indicates poor integrity of the cell layer and wells with high FITC-dextran permeability were excluded. FITC-dextran was included as internal control in each insert used for permeability studies. Fluorescence was measured using a fluorimetric detector. Radioactivity was measured by scintillation counting. For LC-MS/MS analysis, aliquots (200 μl) from the basal compartment were diluted with an equal volume of 100% acetonitrile containing 0.1% formic acid, centrifuged to remove cell debris, and evaporated under N₂. After reconstitution, samples were analyzed by LC-MS/MS. Mass balance was determined considering the amount of compound recovered in the donor and receiver chamber at the end of the assay relative to the amount added to the donor chamber at time 0.

Permeability was defined as the apparent permeability coefficient (Pₐₚₚ), which is a measure of the appearance rate of the compound in the receiver chamber, expressed in cm/s. Pₐₚₚ is calculated according the following equation:

Pₐₚₚ [cm/sec]=V_{d}*ΔMᵣ/A*M_{d}*Δt, Equation 2:

where

V_{d}=volume in the donor compartment in cm³ or mL;

ΔMᵣ=total amount of compound in the receiver compartment after t seconds;

M_{d}=donor amount (added at time 0)

Δt=time measured in seconds

A=filter area in cm² (for 24 well plate, A=0.7 cm²).

To correct for the contribution of filter and substrate, Pₐₚₚ was also determined for the cell-free system. Permeability of the endothelial cell layer was determined using the following equation:

1/Pₑ=1/(Pₜₒₜₐₗ−(1/P_{f}), Equation 3:

where

Pₜₒₜₐₗ=the P of the total system,

P_{f}=P for the cell-free filter, and

Pₑ═P for the endothelial cell layer alone. In this equation, the total resistance of the system towards passage of a substance is additively composed of two parallel resistances: that of the cell monolayer and that of the filter.

TABLE 7
In vitro apparent permeability (Pₐₚₚ) in the blood-brain barrier model
of porcine brain endothelial cells, co-cultured with rat astrocytes
(PBECs/As).

| | Pₐₚₚ (×10⁻⁶ cm/s) | |
|---|---|---|
| Compound No. | Diastereoisomer A ^{a} | Diastereoisomer B |
| R1006 | 3.17 | 3.18 |
| R1007 | 3.1 | 3.6 |

^{a} In all cases, “Diastereoisomer A” refers to the upfield shift in the ³¹P NMR.

All of the U.S. patents, U.S. patent application publications, U.S. patent applications, foreign patents, foreign patent applications, and non-patent publications referred to in this specification, and/or listed in the Application Data Sheet, including U.S. Provisional Patent Application No. 62/366,428 filed on Jul. 25, 2016, are incorporated herein by reference, in their entirety. Aspects of the embodiments can be modified, if necessary to employ concepts of the various patents, applications, and publications to provide yet further embodiments.

While specific embodiments of the invention have been illustrated and described, it will be readily appreciated that the various embodiments described above can be combined to provide further embodiments, and that various changes can be made therein without departing from the spirit and scope of the invention. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following structure (I): 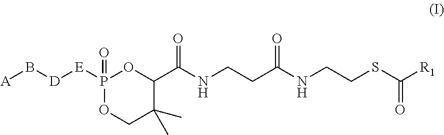
or a pharmaceutically acceptable salt thereof, wherein:
E is O or NR₂;
D is absent, C₁-C₃ alkylene, C(O)O(alkylene) or aryl, wherein each of said C₁-C₃ alkylene, C(O)O(alkylene) and aryl is unsubstituted or substituted with R₃;
B is absent, C₁-C₃ alkylene, C₃-C₆ cycloalkylene, (C₁-C₃ alkylene)NR₂, C(O)NR₂(alkylene), aryl, heteroaryl or heterocyclyl, wherein each of said C₁-C₃ alkylene, C₃-C₆ cycloalkylene, C(O)NR₂(alkylene), aryl, heteroaryl and heterocyclyl is unsubstituted or substituted with R₆;
A is absent, H, OR₅, R₅C(O), R₅OC(O), R₅OC(O)O, R₅C(O)O, R₅C(O)S, NR₂R₅C(O), NR₂R₅C(O)O, R₅C(O)NR₂, C(O)ONR₂, S(O)NR₂, R₅SO₂NR₂, NR₂R₅, C₁-C₆ alkyl, C₃-C₆ cycloalkyl, heterocyclyl, aryl or heteroaryl, wherein each of said C₁-C₆ alkyl, C₃-C₆ cycloalkyl, heterocyclyl, aryl and heteroaryl is unsubstituted or substituted with R₆;
R₁ is H, C₁-C₆ alkyl, aryl, arylalkyl, heteroaryl, heteroarylalkyl, heterocyclyl, heterocyclylalkyl, C₃-C₆ cycloalkyl, or cycloalkylalkyl, wherein each of said C₁-C₆ alkyl, aryl, arylalkyl, heteroaryl, heteroarylalkyl, heterocyclyl, heterocyclylalkyl, C₃-C₆ cycloalkyl, and cycloalkylalkyl is unsubstituted or substituted with R₆;
R₂ is H or C₁-C₆ alkyl;
R₃ is H, C₁-C₆ alkyl, hydroxy, amino, arylalkyl, heteroarylalkyl or C₃-C₆ cycloalkyl, wherein each of said C₁-C₆ alkyl, arylalkyl, heteroarylalkyl and C₃-C₆ cycloalkyl is unsubstituted or substituted with R₄;
R₄ is C₁-C₆ alkyl, C₁-C₆ alkoxy, hydroxy or amino;
R₅ is H, C₁-C₆ alkyl, C₁-C₆ alkoxy, aryl, arylalkyl, heteroaryl, heteroarylalkyl, heterocyclyl, heterocyclylalkyl, C₃-C₆ cycloalkyl, cycloalkylalkyl, amino, alkylamino, dialkylamino, aminoalkyl, alkylaminoalkyl or dialkylaminoalkyl, wherein each of said C₁-C₆ alkyl, C₁-C₆ alkoxy, aryl, arylalkyl, heteroaryl, heteroarylalkyl, heterocyclyl, heterocyclylalkyl, amino, alkylamino, dialkylamino, aminoalkyl, alkylaminoalkyl and dialkylaminoalkyl is unsubstituted or substituted with R₆;
R₆ is C₁-C₆ alkyl, C₁-C₆ alkoxy, hydroxyl, amino, halo, oxo, CN, NO₂, SF₅, heterocyclyl, heterocyclylalkyl, aryl, arylalkyl, C₃-C₆ cycloalkyl, C₃-C₄ spiro-substituted cycloalkyl, cycloalkylalkyl, SO₂R₇, R₇C(O), R₇C(O)NR₂ or C(O)OR₈, wherein each of said C₁-C₆ alkyl, C₁-C₆ alkoxy, heterocyclyl, heterocyclylalkyl, aryl, arylalkyl, C₃-C₆ cycloalkyl, C₃-C₄ spiro-substituted cycloalkyl and cycloalkylalkyl is unsubstituted or substituted with R₇;
R₇ is C₁-C₆ alkyl, C₁-C₆ alkoxy, hydroxyl, halo, oxo, CN, NO₂, SF₅, amino, alkylamino or dialkylamino; and
R₈ is H, C₁-C₆ alkyl or arylalkyl; or
D is absent, and A, B, and E together form a 6-membered heterocyclic or heteroaryl ring, wherein said heterocyclic or heteroaryl ring is unsubstituted or substituted with R₆.

2. A compound according to claim 1, wherein R₁ is C₁-C₆ alkyl.

3. (canceled)

4. A compound according to claim 1, wherein E is O.

5. A compound according to claim 4, wherein D is absent, C₁-C₃ alkylene or C(O)O(alkylene).

6.-11. (canceled)

12. A compound according to claim 4, wherein B is absent, (C₁-C₃ alkylene)NR₂ or (C₁-C₃ alkylene)NR₂ substituted with R₆.

13.-19. (canceled)

20. A compound according to claim 4, wherein A is R₅OC(O), R₅OC(O)O, R₅C(O)O, R₅C(O)S, aryl, heteroaryl, aryl substituted with R₆ or heteroaryl substituted with R₆.

21.-29. (canceled)

30. A compound according to claim 1, wherein:
E is O;
D is C₁-C₃ alkylene or C(O)O(alkylene);
B is absent, (C₁-C₃ alkylene)NR₂ or (C₁-C₃ alkylene)NR₂ substituted with R₆;
A is OR₅, R₅C(O)O, R₅C(O)S, aryl, heteroaryl, aryl substituted with R₆ or heteroaryl substituted with R₆;
R₁ is C₁-C₆ alkyl;
R₂ is H;
R₅ is C₁-C₆ alkyl, C₁-C₆ alkyl substituted with R₆, aryl substituted with R₆ or heteroaryl substituted with R₆;
R₆ is C₁-C₆ alkyl, C₁-C₆ alkyl substituted with R₇, C₁-C₆ alkoxy, C(O)OR₈, amino or halo; and
R₇ is halo; and
R₈ is arylalkyl.

31. A compound according to claim 30, wherein:
D is C(O)O(alkylene);
B is absent;
A is heteroaryl substituted with R₆; and
R₆ is C₁-C₆ alkyl.

32. A compound according to claim 30, wherein:
D is C(O)O(alkylene);
B is (C₁-C₃ alkylene)NR₂;
A is OR₅C(O)S;
R₅ is C₁-C₆ alkyl; and
R₆ is C(O)OR₈.

33. A compound according to claim 30, wherein:
D is C(O)O(alkylene);
B is (C₁-C₃ alkylene)NR₂;
A is R₅C(O)O; and
R₅ is C₁-C₆ alkyl.

34. A compound according to claim 1, wherein E is NR₂.

35.-50. (canceled)

51. A compound according to claim 1, wherein the compound is

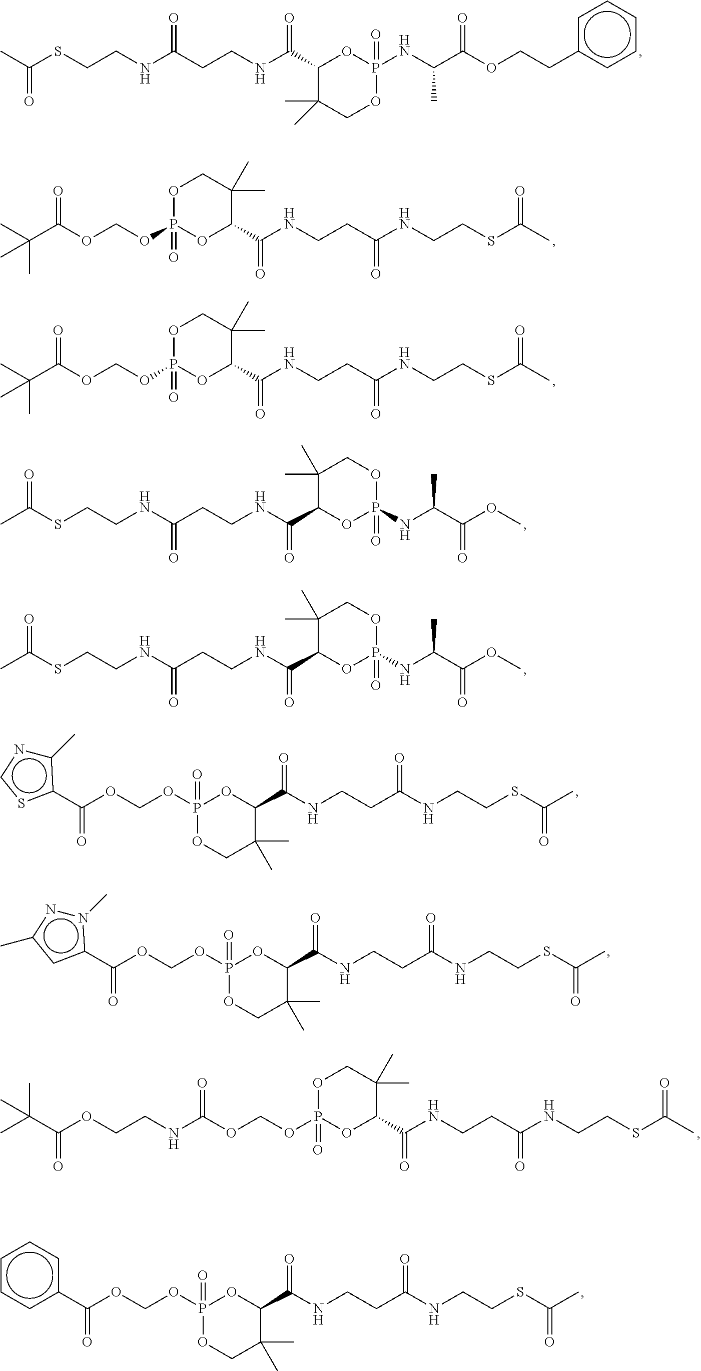

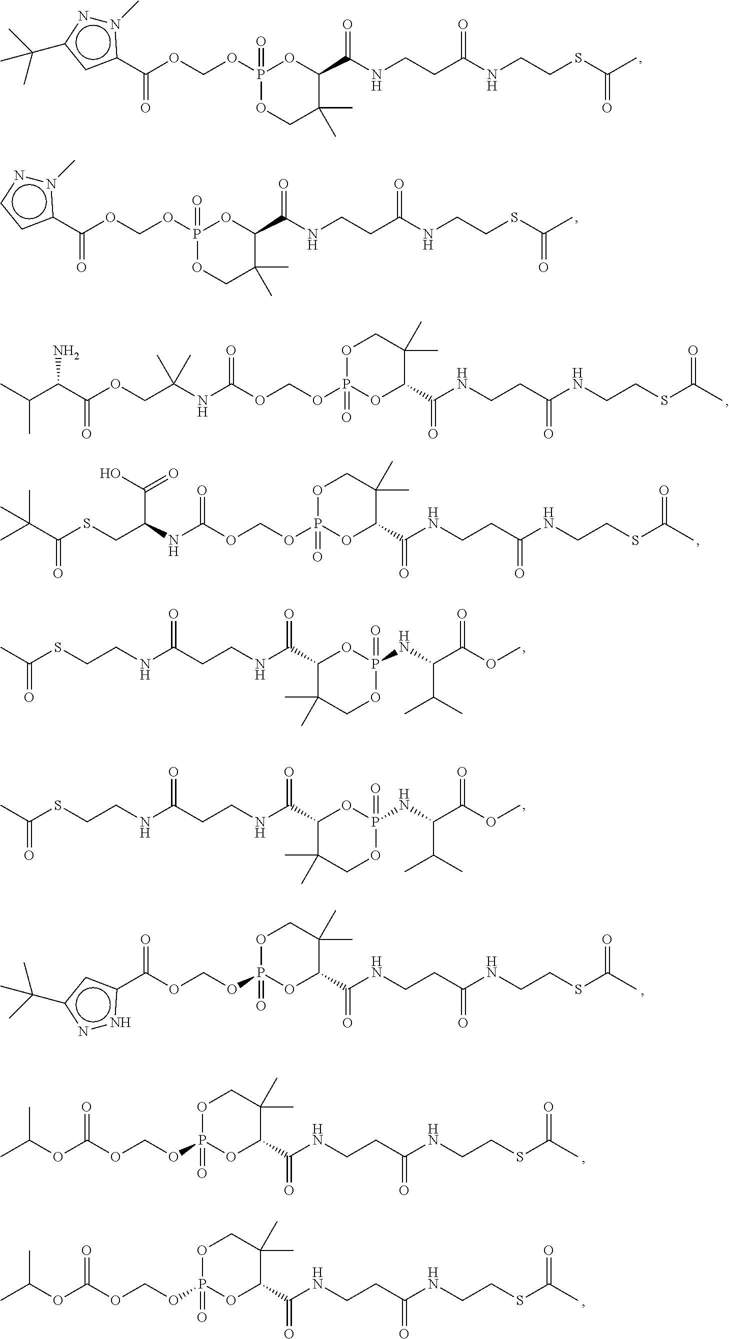

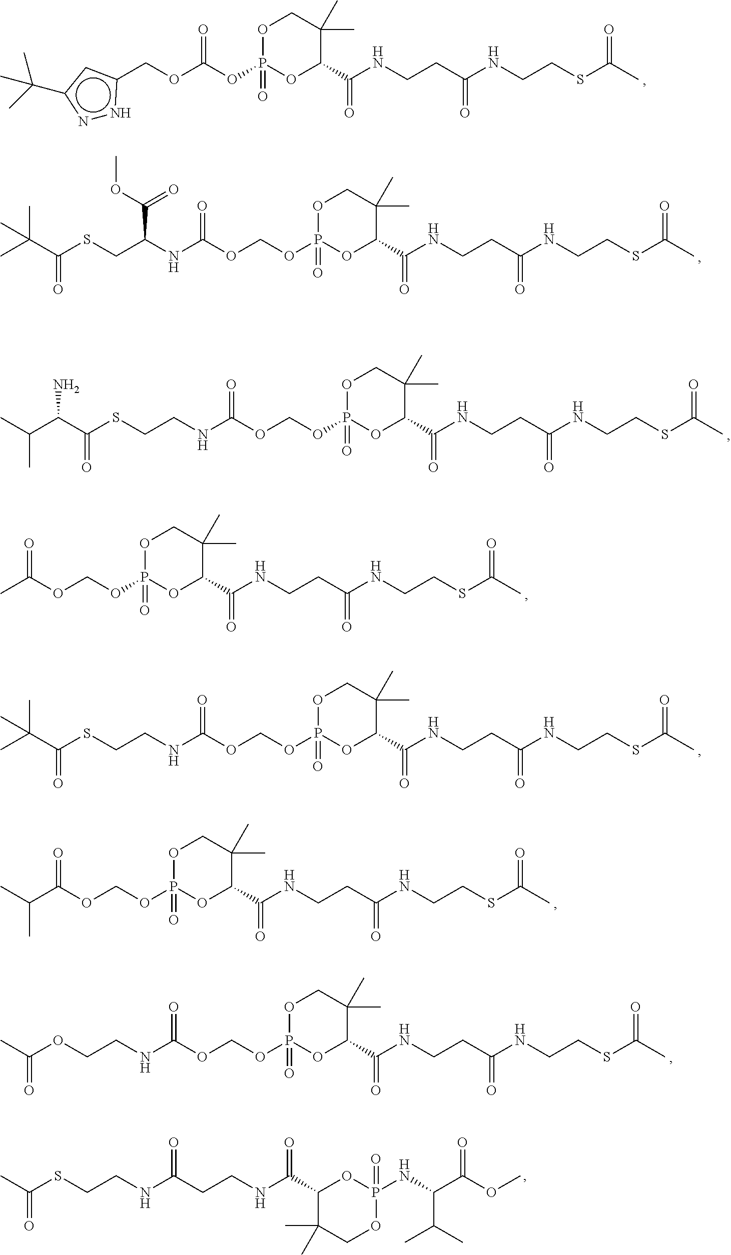

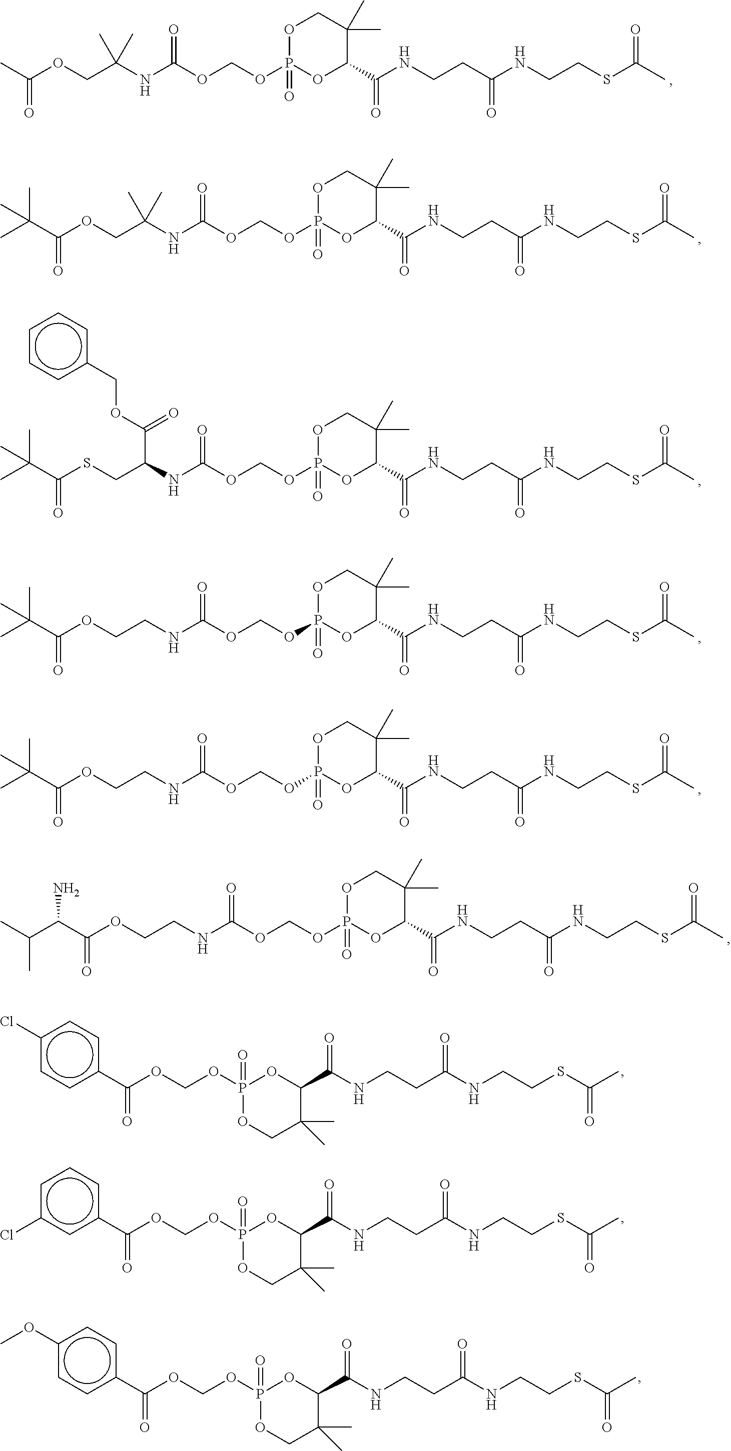

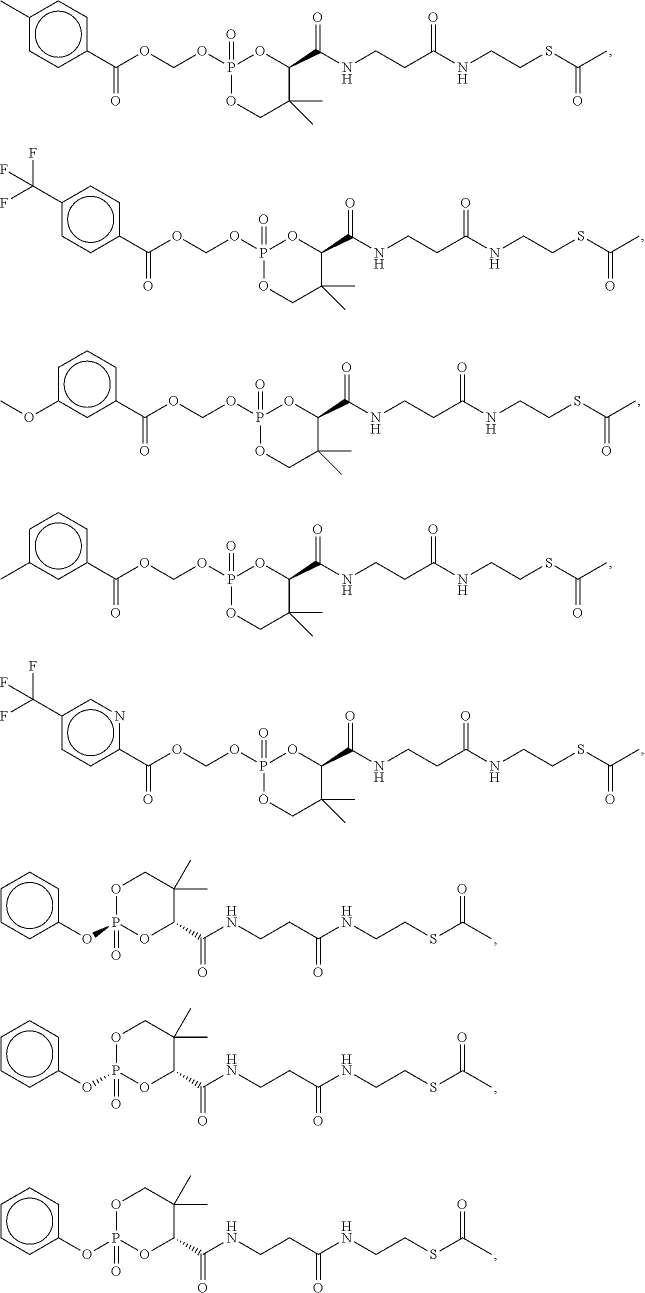

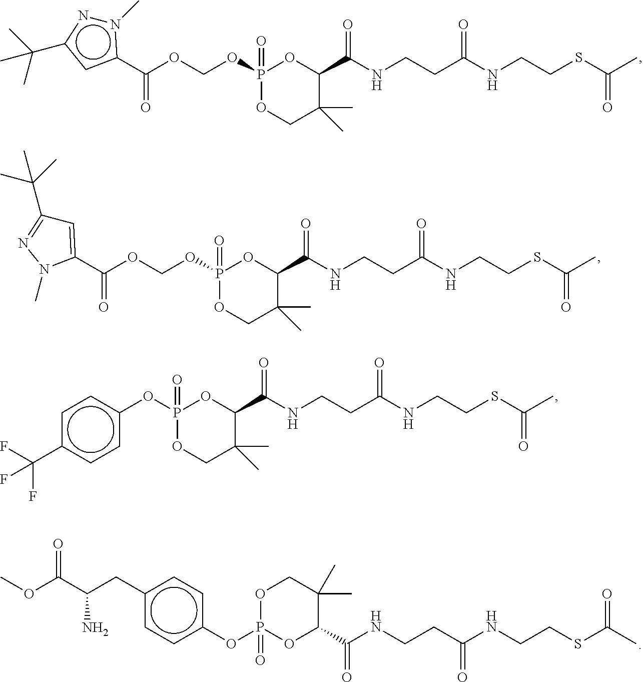

52. A pharmaceutical composition comprising a compound of claim 1, or a pharmaceutically acceptable salt thereof, and a pharmaceutically acceptable carrier, diluent or excipient.

53. A method of increasing 4′-phosphopantetheine production in a subject in need thereof, the method comprising administering a compound of claim 1 to the subject.

54. (canceled)

55. A method of treating a subject having a disorder associated with pantothenate kinase enzyme deficiency comprising administering a compound of claim 1 to a subject in need thereof.

56. The method of claim 55, wherein said disorder is pantothenate kinase-associated neurodegeneration, 4′-phosphopantothenic acid deficiency, neurodegeneration with brain iron accumulation or a pantothenate kinase gene (PANK) defect.

57.-59. (canceled)

60. The method of claim 56, wherein said PANK gene defect comprises a PANK1 gene defect, a PANK2 gene defect, a PANK3 gene defect or a PANK4 gene defect.

61.-63. (canceled)

64. A method of treating a subject having a disorder associated with Coenzyme A deficiency, a condition associated with abnormal neuronal function, a condition associated with neuronal cell iron accumulation, neurodegeneration with brain iron accumulation, a disorder associated with deficiency of 4′-phosphopantothenoylcysteine synthase, or a disorder associated with deficiency of 4′-phosphopantothenoylcysteine decarboxylase, comprising administering a compound of claim 1 to a subject in need thereof.

65.-70. (canceled)

71. The method of claim 56, wherein the compound or composition is administered to the subject three times a day.

72. The method of claim 56, wherein the compound or composition is administered to the subject three times a day for a period of one to four weeks, and then two times a day or one time a day for a period of greater than or equal to 12 weeks.
